(19) United States
(12) Patent Application Publication
(10) Pub. No.: US 2016/0237158 A1
(43) Pub. Date: Aug. 18, 2016

(54) Methods and Compositions for Targeting Tumor Microenvironment and for preventing Metastasis

(71) Applicants: INSTITUT NATIONAL DE LA SANTÉ ET DA LA RECHERCHE MÉDICALE (INSERM), Paris (FR); CENTRE NATIONAL DE LA RECHERCHE SCIENTIFIQUE (CNRS), Paris (FR); UNIVERSITE NICE SOPHIA ANTIPOLIS, Nice (FR)

(72) Inventors: Cedric GAGGIOLI, Nice Cedex 2 (FR); Guerrino MENEGUZZI, Nice Cedex 2 (FR); Jean ALBRENGUES, Nice Cedex 2 (FR)

(21) Appl. No.: 15/023,806

(22) PCT Filed: Sep. 23, 2014
(86) PCT No.: PCT/EP2014/070271

§ 371 Date: Mar. 22, 2016

(30) Foreign Application Priority Data
Sep. 23, 2013 (EP) 13306298.4

(51) Int. Cl. C07K 16/28; C12Q 1/68; A61K 31/7064; G01N 33/574; A61K 31/519; C07K 16/24

(52) U.S. Cl. CPC C07K 16/2866; A61K 31/519; C07K 16/244; C07K 16/28; A61K 31/7064; G01N 33/57488; C12Q 1/6886; G01N 33/57496; G01N 2333/5415; C12Q 2600/158; C12Q 2600/106; G01N 2333/4706; C07K 2317/76

(57) ABSTRACT

The invention relates to the invention relates to methods and compositions for targeting tumor microenvironment (TME) and for preventing metastasis. The invention also relates to methods and compositions for inhibiting the pro-invasive stromal fibroblast activity as well as the pro-invasive stromal fibroblast activation in a patient in need thereof such as a patient affected with a solid cancer.

### FIELD OF THE INVENTION

The invention relates generally to the field of oncology. More specifically, the invention relates to methods and compositions for targeting tumor microenvironment (TME) and for preventing metastasis. The invention also relates to methods and compositions for inhibiting the pro-invasive stromal fibroblast activity as well as the pro-invasive stromal fibroblast activation in a patient in need thereof such as a patient affected with a solid cancer.

### BACKGROUND OF THE INVENTION

Malignant evolution of solid cancers relies on complex cell-to-cell interactions sustained by a broad network of physical and chemical mediators that constitutes the tumour microenvironment′. Such a cellular network involves both tumour and non-tumour cells embedded in a modified extracellular matrix (ECM) rich in growth factors, chemokines and cytokines that supports cancer cell growth and invasive spreading. Carcinoma associated fibroblasts (CAF) are the most representative non-cancer cells within the tumour microenvironment^{3,4}, and their presence is associated with poor clinical prognosis⁵⁻⁹. It is clear that under the influence of bioactive molecules within the tumour stroma, resident fibroblasts are activated and promote tumourigenesis¹⁰⁻¹⁴. Indeed, CAF can support tumour-initiation¹⁵, -inflammation¹⁶ and -angiogenesis′. CAF are also responsible for pro-invasive ECM remodelling and track formation leading to collective carcinoma cell invasion¹⁸.

Inflammation is hallmark of cancer progression^{1,19}. It is established that paracrine secretion of multiples molecules, including TGFβ, growth factors, and pro-inflammatory molecules such as Interleukin-6 (IL6) family cytokines, by cancer cells, promotes tumourigenesis^{2,20}. TGFβ-family cytokines are known to drive myofibroblast activation during wound healing and cancer progression^{21,22}, but the role of stroma-specific TGFβ-dependent signalling during cancer invasion remains however unclear. Indeed, specific stromal deletion of TGFβ-receptor (TGFβr) II in mice promotes invasive tumourigenesis²³⁻²⁵. Similarly, pharmacological inhibition of TGFβr I in a mice model of chemically-induced carcinoma development represses papilloma progression towards development of aggressive carcinoma²⁶. The role of TGFβ-dependent signalling in cancer promotion is multi-faceted and targeting TGFβ signalling in patients has been so far deceiving²⁷.

Therefore, understanding how cancer cells induce a fibroblast-dependent pro-invasive tumour microenvironment as well as understanding the mechanisms of ECM remodelling and its regulation provides key issues for prognosis and treatment of patients with solid cancers.

### SUMMARY OF THE INVENTION

In a first aspect, the invention relates to an inhibitor of the Janus Kinase 1 (JAK1)/Signal Transducer and Activator of Transcription 3 (STAT3) signalling pathway for use in preventing metastasis in a patient affected with a Leukemia Inhibitory Factor (LIF)-expressing solid cancer.

In a second aspect, the invention also relates to an inhibitor of the JAK1/STAT3 signalling pathway for use in inhibiting the pro-invasive stromal fibroblast activity of carcinoma-associated fibroblasts (CAFs) in a patient affected with LIF-expressing solid cancer.

In a third aspect, the invention also relates to a LIF antagonist for use in inhibiting the pro-invasive stromal fibroblast activation of CAFs in a patient affected with a LIF-expressing solid cancer.

In a fourth aspect, the invention also relates to a method for determining whether a patient is likely to benefit from a therapy with an inhibitor of the JAK1/STAT3 signalling pathway and/or a LIF antagonist, comprising a step of determining the expression level of LIF in a biological sample obtained from said patient.

In a fifth aspect, the invention relates to an inhibitor of the JAK1/STAT3 signalling pathway and/or a LIF antagonist for use in treating recessive dystrophic epidermolysis bullosa (RDEB).

In a sixth aspect, the invention relates to an inhibitor of the JAK1/STAT3 signalling pathway and/or a LIF antagonist for use in preventing metastasis in a patient affected with RDEB.

In a seventh aspect, the invention relates to a pharmaceutical composition or a kit-of-parts composition comprising inhibitor of the JAK1/STAT3 signalling pathway, a LIF antagonist and a DNA methyltransferase (DNMT) inhibitor.

In a eight aspect, the invention also relates to a pharmaceutical composition or a kit-of-parts composition comprising an inhibitor of the JAK1/STAT3 signalling pathway and a LIF antagonist for use in preventing metastasis or for use in improving survival time of patient in need thereof.

In a ninth aspect, the invention also relates to a pharmaceutical composition or a kit-of-parts composition comprising an inhibitor of the JAK1/STAT3 signalling pathway and a DNMT inhibitor for use in preventing metastasis or for use in improving survival time in patient in need thereof.

### DETAILED DESCRIPTION OF THE INVENTION

The inventors focused their research on the determination of the signalling crosstalk between tumour cells and fibroblasts conferring pro-invasive properties to the tumour microenvironment. The objective of the invention is indeed to propose new therapeutics approaches for the treatment of cancer, in particular aggressive tumours and metastases.

The inventors have thus identified Leukemia Inhibitory Factor (LIF) as a tumour promoter that mediates pro-invasive activation of stromal fibroblasts independent of alpha-Smooth Muscle Actin (αSMA) expression. They demonstrated that a pulse of TGFβ establishes stable and pro-invasive fibroblast activation by inducing LIF production in both fibroblasts and tumour cells. LIF signalling in fibroblasts mediates TGFβ-dependent acto-myosin contractility and ECM remodelling, resulting in collective carcinoma cell invasion in vitro and in vivo. Accordingly, carcinomas from multiple origin and melanomas display strong LIF upregulation, which correlates with dense collagen fibres organisation in breast and skin carcinomas. Blockade of JAK activity by the ruxolitinib inhibitor counteracts fibroblast-dependent collective carcinoma cell invasion in vitro and in vivo. These findings make LIF a pro-invasive fibroblast producer independent of αSMA and may open novel therapeutic perspectives for patients presenting aggressive primary tumours. Indeed, deciphering the role of TGFβ signalling in tumourigenesis reveals a distinct but collaborative role for TGFβ and LIF cytokines TGFβ drives phenotypic fibroblast conversion into carcinoma-associated fibroblasts (CAF)-like cells, while LIF supports formation of a pro-invasive tumour microenvironment.

The inventors have also shown that a combination of a JAK1 inhibitor (ruxolitinib) and a DNA methyltransferase (DNMT) inhibitor (azacytidine) is useful for preventing metastasis at long-term in a patient in need thereof by a constitutive inhibition of invasiveness of stomal cells such as CAF following their reprogrammation in healthy fibroblasts.

### Therapeutic Methods of the Invention

Pro-invasive properties are conferred to fibroblasts via activation of the JAK1/STAT3 pathway by TGFβ1 and LIF. Thus, the inhibition of pro-invasive stromal fibroblast activity and also the pro-tumorigenic ECM remodeling (by inhibiting track formation leading to collective carcinoma cell invasion) and thereby the prevention of metastasis in a patient affected with a solid tumor. Thus, the prevention of metastasis entails using a therapeutically effective amount of a JAK1/STAT3 pathway inhibitor to inhibit or reduce JAK1 kinase activity or STAT3 phosphorylation, and preferably to inhibit or reduce JAK1 kinase activity.

In a first aspect, the invention relates to inhibitor of the Janus Kinase 1 (JAK1)/Signal Transducer and Activator of Transcription 3 (STAT3) signalling pathway for use in preventing metastasis in a patient in need thereof.

In one embodiment, the patient in need thereof is a patient affected with a Leukemia Inhibitory Factor (LIF)-expressing solid cancer.

In one embodiment, metastasis is induced by the pro-invasive stromal fibroblast activity of SMA expressing fibroblasts (α-SMA+ fibroblasts). Such activated α-SMA+ fibroblasts are known as to carcinoma-associated fibroblasts (CAFs).

In another embodiment, metastasis is induced by the pro-invasive stromal fibroblast activity of non-SMA expressing fibroblasts (α-SMA-fibroblasts). Such activated α-SMA-fibroblasts also display a pro-invasive activity as disclosed herein.

As used herein, the term “preventing” a disorder or a condition refers to keeping from occurring, or to hinder, defend from, or protect from the occurrence of a disorder or a condition or phenotype, including a symptom.

By “metastasis” or “tumour metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream or lymphatics, and stopping at a distant site. At the new site, the cells establish a blood supply and can grow to form a life-threatening mass. In certain embodiments, the term metastatic tumor refers to a tumor that is capable of metastasizing, but has not yet metastasized to tissues or organs elsewhere in the body. In certain embodiments, the term metastatic tumor refers to a tumor that has metastasized to tissues or organs elsewhere in the body.

As used herein, the term “patient in need thereof” refers to a mammal, such as a rodent, a feline, a canine, and a primate. Preferably, a patient according to the invention is a human. In one embodiment, the patient in need thereof may be at risk of developing metastasis (e.g. a patient affected with a solid cancer such as an epithelial cancer or a patient affected with recessive dystrophic epidermolysis bullosa (RDEB)).

As used herein, the term “cancer” or “tumor” refer to the pathological condition in mammals that is typically characterized by unregulated cell growth. Examples of cancer include, but are not limited to, carcinoma, lymphoma, blastoma, sarcoma, and leukemia. Within the context of the invention, the term “cancer” refers to solid cancer and/or conditions associated with activated stroma, including carcinoma epithelial cell cancers. Epithelial cell cancers comprise approximately 80-85% of all cancers, and include, amongst others, breast, bladder, lung, pancreatic, thyroid and prostate cancers. Here, examples of solid cancer include, head and neck cancer, esophageal cancer, thyroid cancer, small cell lung cancer, non-small cell lung cancer, breast cancer, stomach cancer, liver cancer, bladder cancer, prostate cancer, skin cancer and melanoma (including malignant melanoma).

In a preferred embodiment, the cancer is a Leukemia Inhibitory Factor (LIF)-expressing solid cancer.

As used herein, the term “recessive dystrophic epidermolysis bullosa” (RDEB) refers to a rare genetic skin blistering disorder caused by mutations in the collagen VII gene. RDEB is associated with recurrent blister formation, soft tissue scarring and mitten deformities, and development of aggressive squamous cell carcinoma. Patients suffering from RDEB are prone to develop aggressive and metastatic skin cancers with fatal demise. Indeed, squamous cell carcinoma (SCC) tumors arise at the site of long-term blistering due to the lack of collagen VII expression, which causes separation of the epidermis from dermis with a cleavage beneath the lamina densa. Analysis of RDEB human and murine skin has demonstrated the presence of pro-inflammatory molecules at the blister area, which correlates with extracellular matrix remodelling, and presence of activated fibroblasts (CAF).

The term “JAK1/STAT3” signalling pathway is used throughout the specification to identify a cellular signaling pathway utilizing Janus Kinase (JAK) proteins and Signal Transducers and Activators of Transcription (STAT) proteins. JAK proteins are tyrosine kinases which bind to cytokine receptors of a cell and, upon association of an extracellular ligand, become activated, phosphorylating phosphotyrosine-binding SH2 domains. STAT proteins, containing the SH2 domains, are activated and dimerize. Dimeric STAT proteins migrate into the nucleus activating transcription of target genes.

In one embodiment, the inhibitor of the JAK1/STAT3 signalling pathway is a JAK1 inhibitor.

As used herein, the term “Janus Kinase 1” (JAK1) is well known in the art and refers to a member of a class of protein-tyrosine kinases (PTK) characterized by the presence of a second phosphotransferase-related domain immediately N-terminal to the PTK domain. The JAK1 gene encodes a 1154 amino acid polypeptide and the naturally occurring human JAK1 protein has an aminoacid sequence as shown in Uniprot Accession number NP 002218.

As used herein, the term “JAK1 inhibitor” refers to a molecule (natural or synthetic) which inhibit signalling through JAK1, as well as compounds which inhibit the expression of the JAK1 gene. They include compounds which inhibit the activity of JAK1, or by inhibiting JAK1 signalling by other mechanisms.

In a particular embodiment, the JAK1 inhibitor is a JAK1/2 inhibitor.

In a preferred embodiment, the JAK1/2 inhibitor is a heteroaryl substituted pyrrolo[2,3-b]pyridine such as those disclosed in the PCT application No. WO 2007/070514.

Accordingly, the JAK1/2 inhibitor is a compound of formula (I):

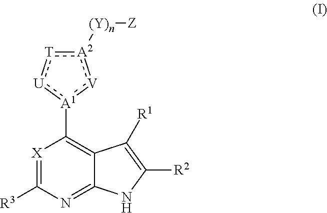

including pharmaceutically acceptable salt forms or prodrugs thereof, wherein:
A¹ and A² are independently selected from C and N;
T, U, and V are independently selected from O, S, N, CR⁵, and NR⁶;

wherein the 5-membered ring formed by A′, A², U, T, and V is aromatic;
X is N or CR⁴;
Y is C₁₋₈ alkylene, C₂₋₈ alkenylene, C₂₋₈ alkynylene, (CR¹¹R¹²)ₚ—(C₃₋₁₀ cycloalkylene-(CR¹¹R¹²)_{q}, (CR¹¹R¹²)ₚ-(arylene)-(CR¹¹R¹²)_{q}, (CR¹¹R¹²)ₚ—(C₁₋₁₀ heterocycloalkylene)-(CR¹¹R¹²)_{q}, (CR¹¹R¹²)ₚ-(heteroarylene)-(CR¹¹R¹²)_{q}, (CR¹¹R¹²)ₚO(CR¹¹R¹²)_{q}, (CR¹¹R¹²)ₚS(CR¹¹R¹²)_{q}, (CR¹¹R¹²)ₚC(O)(CR¹¹R¹²)_{q}, (CR¹¹R¹²)ₚC(O)NR^{c}(CR¹¹R¹²)_{q}, (CR¹¹R¹²)ₚC(O)O(CR¹¹R¹²)_{q}, (CR¹¹R¹²)ₚOC(O)(CR¹¹R¹²)_{q}, (CR¹¹R¹²)ₚOC(O)NR^{c}(CR¹¹R¹²)_{q}, (CR¹¹R¹²)ₚNR^{c}(CR¹¹R¹²)_{q}, (CR¹¹R¹²)ₚNR^{c}C(O)NR^{d}(CR¹¹R¹²)_{q}, (CR¹¹R¹²)ₚS(O)(CR¹¹R¹²)_{q}, (CR¹¹R¹²)ₚS(O)NR^{c}(CR¹¹R¹²)_{q}, (CR¹¹R¹²)ₚS(O)₂(CR¹¹R¹²)_{q}, or (CR¹¹R¹²)ₚS(O)₂NR^{c}(CR¹¹R¹²)_{q}, wherein said C₁₋₈ alkylene, C₂₋₈ alkenylene, C₂₋₈ alkynylene, cycloalkylene, arylene, heterocycloalkylene, or heteroarylene, is optionally substituted with 1, 2, or 3 substituents independently selected from -D¹-D²-D³-D⁴;
Z is H halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, ═C—Rⁱ, ═N—Rⁱ, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, S(O)R^{b}, S(O)NR^{c}R^{d}, S(O)₂R^{b}, NR^{c}S(O)₂R^{b}, C(═NOH)R^{b}, C(═NO(C₁₋₆alkyl)R^{b}, and S(O)₂NR^{c}R^{d}, wherein said C₁₋₈ alkyl, C₂₋₈ alkenyl, or C₂₋₈ alkynyl, is optionally substituted with 1, 2, 3, 4, 5, or 6 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO², OR^{a}, SR^{a}, C(O)R^{b}, C(O)NR^{c}R^{d}, C(O)OR^{a}, OC(O)R^{b} OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, S(O)R^{b}, S(O)NR^{c}R^{d}, S(O)₂R^{b}, NR^{c}S(O)₂R^{b}, C(═NOH)R^{b}, C(═NO(C₁₋₆alkyl))R^{b}, and S(O)²NR^{c}R^{d};
wherein when Z is H, n is 1;
or the —(Y)ₙ—Z moiety is taken together with i) A² to which the moiety is attached, ii) R³ or R⁶ of either T or V, and iii) the C or N atom to which the R⁵ or R⁶ of either T or V is attached to form a 4- to 20-membered aryl, cycloalkyl, heteroaryl, or heterocycloalkyl ring fused to the 5-membered ring formed by A¹, A², U, T, and V, wherein said 4- to 20-membered aryl, cycloalkyl, heteroaryl, or heterocycloalkyl ring is optionally substituted by 1, 2, 3, 4, or 5 substituents independently selected from —(W)ₘ-Q;
W is C₁₋₈alkylenyl, C₂₋₈alkenylenyl, C₂₋₈alkynylenyl, O, S, C(O), C(O)NR^{c′}, C(O)O, OC(O), OC(O)NR^{c′}, NR^{c′}, NR^{c′}C(O)NR^{d′}, S(O), S(O)NR<0′>, S(O)₂, or S(O)₂NR^{c′};
Q is H, halo, CN, NO₂, C₁₋₈ alkyl, C₂₋₈ alkenyl, C₂₋₈ alkynyl, C₁₋₈haloalkyl, halosulfanyl, aryl, cycloalkyl, heteroaryl, or heterocycloalkyl, wherein said C₁₋₈alkyl, C₂₋₈ alkenyl, C₂₋₈ alkynyl, C₁₋₈haloalkyl, aryl, cycloalkyl, heteroaryl, or heterocycloalkyl is optionally substituted with 1, 2, 3 or 4 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy², CN, NO₂, OR^{a′}, SR^{a′}, C(O)R^{b′}, C(O)NR^{c′}R^{d′}, C(O)OR^{a′}, OC(O)R^{b′}, OC(O)NR^{c′}R^{d′}, NR^{c′}R^{d′}, NR^{c′}C(O)R^{b′}, NR^{c′}C(O)NR^{c′}R^{d′}, NR^{c′}C(O)OR^{a′}, S(O)R^{b′}, S(O)NR^{c′}R^{d′}, S(O)₂R^{b′}, NR^{C′}S(O)₂R^{b′}, and S(O)₂NR^{c′}R^{d′};
Cy¹ and Cy² are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, CN, NO₂, OR^{a″}, SR^{a″}, C(O)R^{b″}, C(O)NR^{c″}R^{d″}, C(O)OR^{a″}, OC(O)R^{b″}, OC(O)NR^{c″}R^{d″}, NR^{c″}R^{d″}, NR^{c″}C(O)R^{b″}, NR^{c″}C(O)OR^{a″}, NR^{c″}S(O)R^{b″}; NR^{c″}S(O)₂R^{b″}; S(O)R^{b″}, S(O)NR^{c″}R^{d″}, S(O)₂R^{b″} and S(O)₂NR^{c″}R^{d″};
R¹, R², R³, and R⁴ are independently selected from H, halo, C₁₋₄ alkyl, C₂₋₄ alkenyl, C₂₋₄ alkynyl, C₁₋₄ haloalkyl, halosulfanyl, aryl, cycloalkyl, heteroaryl, heterocycloalkyl, CN, NO₂, OR⁷, SR⁷, C(O)R⁸, C(O)NR⁹R¹⁰, C(O)OR⁷, OC(O)R⁸, OC(O)NR⁹R¹⁰, NR⁹R¹⁰, NR⁹C(O)R⁸, NR⁹C(O)OR⁷, S(O)R⁸, S(O)NR⁹R¹⁰, S(O)₂R⁸, NR⁹S(O)₂R⁸, and S(O)₂NR⁹R¹⁰;
R⁵ is H, halo, C₁₋₄ alkyl, C₂₋₄ alkenyl, C₂₋₄ alkynyl, C₁₋₄ haloalkyl, halosulfanyl CN, NO₂, OR⁷, SR⁷, C(O)R⁸, C(O)NR⁹R¹⁰, C(O)OR⁷, OC(O)R⁸, OC(O)NR⁹R¹⁰, NR⁹R¹⁰, NR⁹C(O)R⁸, NR^{c}C(O)OR⁷, S(O)R⁸, S(O)NR⁹R¹⁰, S(O)₂R, NR⁹S(O)₂R⁸, and S(O)₂NR⁹R¹⁰;
R⁶ is H, C₁₋₄ alkyl, C₂₋₄ alkenyl, C₂₋₄ alkynyl, C₁₋₄ haloalkyl, OR⁷, C(O)R⁸, C(O)NR⁹R¹⁰, C(O)OR⁷, S(O)R⁸, S(O)NR⁹R¹⁰, S(O)₂R⁸, or S(O)₂NR⁹R¹⁰;
R⁷ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R⁸ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R⁹ and R¹⁰ are independently selected from H, C₁₋₁₀ alkyl, C₁₋₆ haloalkyl, C₂₋₆ alkenyl, C₂₋₆ alkynyl, C₁₋₆alkylcarbonyl, arylcarbonyl, C₁₋₆alkylsulfonyl, arylsulfonyl, aryl, heteroaryl, cycloalkyl, heterocycloalkyl, arylalkyl, heteroarylalkyl, cycloalkylalkyl and heterocycloalkylalkyl;
or R⁹ and R¹⁰ together with the N atom to which they are attached form a 4-, 5-, 6- or 7-membered heterocycloalkyl group;
R¹¹ and R¹² are independently selected from H and -E¹-E²-E³-E⁴;
D¹ and E¹ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxyamino, C₁₋₆ alkylamino, and C₂₋₈ dialkylamino;
D² and E² are independently absent or independently selected from C₁₋₆ alkylene, C₂₋₆ alkenylene, C₂₋₆ alkynylene, (C₁₋₆ alkylene)ᵣ-O—(C₁₋₆alkylene)ₛ, (C₁₋₆ alkylene)ᵣ-S—(C₁₋₆alkylene)ₛ, (C₁₋₆ alkylene)ᵣ-NR^{e}—(C₁₋₆ alkylene)ₛ, (C_{1.6} alkylene)ᵣ-CO—(C₁₋₆ alkylene)ₛ, (C_{1.6} alkylene)ᵣ-COO—(C₁₋₆ alkylene)ₛ, (C_{1.6} alkylene)ᵣ-CONR^{e}—(C₁₋₆ alkylene)ₛ, (C_{1.6} alkylene)ᵣ-SO—(C₁₋₆ alkylene)ₛ, (C_{1.6} alkylene)ᵣ-SO₂—(C₁₋₆ alkylene)ₛ, (C_{1.6} alkylene)ᵣ-SONR^{e}—(C₁₋₆ alkylene)ₛ, and (C_{1.6} alkylene)ᵣ-NR^{e}CONR^{e}—(C₁₋₆ alkylene)ₛ, wherein each of the C₁₋₆ alkylene, C₂₋₆ alkenylene, and C₂₋₆ alkynylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxyamino, C₁₋₆ alkylamino, and C₂₋₈ dialkylamino;
D³ and E³ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, C₁₋₆ alkoxy, C₁₋₆ haloalkoxy, amino, C₁₋₆ alkylamino, and C₂₋₈ dialkylamino;
D⁴ and E⁴ are independently selected from H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, S(O)R^{b}, S(O)NR^{c}R^{d}, S(O)₂R^{b}, NR^{c}S(O)₂R^{b}, C(═NOH)R^{b}, C(═NO(C₁₋₆alkyl)R^{b}, and S(O)₂NR^{c}R^{d}, wherein said C₁₋₈alkyl, C₂₋₈ alkenyl, or C₂₋₈ alkynyl, is optionally substituted with 1, 2, 3, 4, 5, or 6 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NRⁱ)NR^{c}R^{d}, NR^{c}C(═NRⁱ)NR^{c}R^{d}, S(O)R^{b}, S(O)NR^{c}R^{d}, S(O)₂R^{b}, NR^{c}S(O)₂R^{b}, C(═NOH)R^{b}, C(═NO(C₁₋₆alkyl))R^{b}, and S(O)₂NR^{c}R^{d};
R^{a} is H, Cy¹, —(C₁₋₆ alkyl)-Cy¹, C₁₋₆ alkyl, C₁₋₆ haloalkyl, C₂₋₆ alkenyl, C₂₋₆ alkynyl, wherein said C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
Rb is H, Cy¹, —(C₁₋₆ alkyl)-Cy¹, C₁₋₆ alkyl, C₁₋₆ haloalkyl, C₂₋₆ alkenyl, C₂₋₆ alkynyl, wherein said C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
R^{a′} and R^{a″} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
R^{b′} and R^{b″} are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
R^{c} and R^{d} are independently selected from H, Cy¹, —(C₁₋₆ alkyl)-Cy¹, C₁₋₁₀alkyl C₁₋₆ haloalkyl, C₂₋₆ alkenyl, C₂₋₆ alkynyl, wherein said C₁₋₁₀ alkyl, C₂₋₆ haloalkyl, C₂₋₆ alkenyl, or C₂₋₆ alkynyl, is optionally substituted with 1, 2, or 3 substituents independently selected from Cy¹, —(C₁₋₆ alkyl)-Cy¹, OH, CN, amino, halo, C₁₋₆ alkyl, C₁₋₆ haloalkyl, and halosulfanyl;
or R^{c′} and R^{d′} together with the N atom to which they are attached form a 4-, 5-, 6- or 7-membered heterocycloalkyl group optionally substituted with 1, 2, or 3 substituents independently selected from Cy¹, —(C₁₋₆ alkyl)-Cy¹, OH, CN, amino, halo, C₁₋₆ alkyl, C₁₋₆ haloalkyl, C₁₋₆ haloalkyl, and halosulfanyl;
R^{c′} and R^{d′} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
or R^{c′} and R^{d′} together with the N atom to which they are attached form a 4-, 5-6- or 7-membered heterocycloalkyl group optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
R^{c″} and R^{d″} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
or R^{c″} and R^{d″} together with the N atom to which they are attached form a 4-, 5-, 6- or 7-membered heterocycloalkyl group optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;

Rⁱ is H, CN, NO₂, or C₁₋₆ alkyl;
R^{e} and R^{f} are independently selected from H and C_{1.6} alkyl;
Rⁱ is H, CN or NO₂;
m is 0 or 1;
n is 0 or 1;
p is 0, 1, 2, 3, 4, 5, or 6;
q is 0, 1, 2, 3, 4, 5 or 6;
r is 0 or 1; and
s is 0 or 1;
wherein when X is N, n is 1, and the moiety formed by A¹, A², U, T, V, and —(Y)ₙ—Z has the formula:

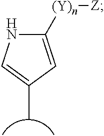

then Y is other than (CR¹¹R¹²)pC(O)NR^{c} (CR¹¹R¹²)_{q}.

In a preferred embodiment, the JAK1 inhibitor is 3-Cyclopentyl-3-[4-(7H-pyrrolo[2,3-d]pyrimidin-4-yl)-1H-pyrazol-1-yl]propanenitrile (INCB018424 or ruxolitinib marketed by Incyte Pharmaceuticals and Novartis for the treatment of intermediate or high-risk myelofibrosis) or a pharmaceutically acceptable salt thereof.

In another embodiment, the JAK1/2 inhibitor is 5-Chloro-N2-[(1S)-1-(5-fluoro-2-pyrimidinyl)ethyl]-N4-(5-methyl-1H-pyrazol-3-yl)-2,4-pyrimidinediamine (or AZD 1480).

In a particular embodiment, the JAK1 inhibitor is a JAK1/3 inhibitor.

In a preferred embodiment, the JAK1/3 inhibitor is 3-[(3R,4R)-4-methyl-3-[methyl(7H-pyrrolo[2,3-d]pyrimidin-4-yl)amino]piperidin-1-yl]-3-oxopropanenitrile (CP-690,550 or tofacitinib) or a pharmaceutically acceptable salt thereof. Tofacitinib, is disclosed in the PCT applications No. WO 01/042246 and WO 02/096909.

In another particular embodiment, the JAK1 inhibitor is JAK1 selective inhibitor.

As used herein, the term “JAK1 selective inhibitor” is a compound that inhibits JAK1 activity preferentially over other Janus kinases. For example, such a compound preferentially inhibits JAK1 over one or more of JAK2, JAK3, and TYK2. Preferably, the compounds inhibit JAK1 preferentially over JAK2 (e.g., have a JAK1/JAK2 IC50 ratio >1).

Specific examples of JAK1 selective inhibitor of the invention, include, but are not limited to, N-(hetero)aryl-pyrrolidine derivatives disclosed in the PCT application No. WO2010/135650 as well as [1,2,4]triazolo[1,5-a]pyridine derivatives (such as GLPG0634) disclosed in the PCT application No. WO2010/010184.

In another particular embodiment, the JAK1 inhibitor is an inhibitor of JAK1 gene expression. An “inhibitor of expression” refers to a natural or synthetic compound that has a biological effect to inhibit the expression of a gene. Accordingly, the inhibitor of JAK1 gene expression is selected form the group consisting of an antisense oligonucleotide, a small inhibitory RNA (siRNA) and a ribozyme.

Inhibitors of JAK1 gene expression for use in the invention may be based on antisense oligonucleotide constructs. Anti-sense oligonucleotides, including anti-sense RNA molecules and anti-sense DNA molecules, would act to directly block the translation of JAK1 mRNA by binding thereto and thus preventing protein translation or increasing mRNA degradation, thus decreasing the level of JAK1, and thus activity, in a cell. For example, antisense oligonucleotides of at least about 15 bases and complementary to unique regions of the mRNA transcript sequence encoding JAK1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). It should be further noted that antisense oligonucleotides may be modified with phosphorothioate to prevent their in vivo hydrolysis by nucleases. Such modifications are well known in the art.

Small inhibitory RNAs (siRNAs) can also function as inhibitors of NF-κB2 gene expression for use in the invention. JAK1 gene expression can be reduced by contacting the tumor, subject or cell with a small double stranded RNA (dsRNA), or a vector or construct causing the production of a small double stranded RNA, such that JAK1 gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; International Patent Nos. WO 01/36646, WO 99/32619, and WO 01/68836).

In one particular embodiment, the sequence of the siRNA targeting JAK1 is GACAUGAUAUUGAGAACGA represented by SEQ ID NO: 1.

In one particular embodiment, the sequence of the siRNA targeting JAK1 is UUACAAGGAUGACGAAGGA represented by SEQ ID NO: 2.

Ribozymes can also function as inhibitors of NF-κB2 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of NF-κB2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNA and ribozymes useful as inhibitors of NF-κB2 gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide, siRNA or ribozyme nucleic acid to the cells and preferably cells expressing cancer cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In another embodiment, the inhibitor of the JAK1/STAT3 signalling pathway is a STAT3 inhibitor.

As used herein, the term “Signal transducer and activator of transcription 3” (STAT3) refers to a member of a family of DNA-binding proteins that plays an important role in cytokine signal transduction. Phosphorylation on Tyr705 results in its activation Two phosphorylated and activated STAT3 monomers dimerize through reciprocal pTyr-SH2 domain interactions, translocate to the nucleus, and bind to specific DNA-response elements of target genes, thereby inducing gene transcription. The STAT3 gene encodes a 770 amino acid polypeptide and the naturally occurring human STAT3 protein has an aminoacid sequence as shown in Uniprot Accession number NP_644805.

As used herein, the term “STAT3 inhibitor” refers to a molecule (natural or synthetic) which inhibit signalling through STAT3, as well as compounds which inhibit the expression of the STAT3 gene. They include compounds which inhibit the activity of STAT3, or by inhibiting STAT3 signalling by other mechanisms. STAT3 inhibitors are well known in the art and are described for instance in Page B D et al., Signal transducer and activator of transcription 3 inhibitors: a patent review. Expert Opin Ther Pat. 2011 January; 21(1):65-83.

Typically, STAT3 inhibitor are WP1066, SPI (a 28-mer peptide, derived from the STAT3 SH2 domain) disclosed in WO 2011/163423, substituted purine analogs (e.g. substituted 2-(9H-purin-9-yl) acetic acid analogues) disclosed in WO 2011/163424, and N-[1,3,4-oxadiazol-2-yl]-4-quinolinecarboxamide derivatives disclosed in WO 2010/004761.

In one embodiment, the STAT3 inhibitor is an inhibitor of STAT3 gene expression.

Therefore, an “inhibitor of STAT3 gene expression” denotes a natural or synthetic compound that has a biological effect to inhibit the expression of STAT3 gene.

In a particular embodiment, the inhibitor of STAT3 gene expression is selected form the group consisting of an antisense oligonucleotide, a small inhibitory RNA (siRNA) and a ribozyme. Accordingly, several approaches for inhibiting STAT3 expression have been disclosed in U.S. Pat. Nos. 5,719,042 and 5,844,082 and in International Patent applications Nos. WO 00/61602, WO 2005/083124 and WO 2012/161806.

In one particular embodiment, the sequence of the siRNA targeting STAT3 is GAGAUUGACCAGCAGUAUA represented by SEQ ID NO: 3.

In one particular embodiment, the sequence of the siRNA targeting STAT3 is CCAACAAUCCCAAGAAUGU represented by SEQ ID NO: 4.

In a second aspect, the invention relates to a Leukemia inhibitory factor (LIF) antagonist for use in preventing metastasis in a patient in need thereof.

In one embodiment, the patient in need thereof is a patient affected with a Leukemia Inhibitory Factor (LIF)-expressing solid cancer.

In one embodiment, metastasis is induced by the pro-invasive stromal fibroblast activity of SMA expressing fibroblasts (α-SMA+ fibroblasts). Such activated α-SMA+ fibroblasts are known as to carcinoma-associated fibroblasts (CAFs).

In another embodiment, metastasis is induced by the pro-invasive stromal fibroblast activity of non-SMA expressing fibroblasts (α-SMA-fibroblasts). Such activated α-SMA-fibroblasts also display a pro-invasive activity as disclosed herein.

As used herein, the term “Leukemia Inhibitory Factor” (LIF) refers to an Interleukin-6 (IL-6)-type cytokine that is involved in a variety of biological activities and has effects on different cell types. Activation of the LIF receptor has been shown to stimulate intracellular tyrosine kinases (especially the JAK/STAT (Janus kinase/signal transducer and activator of transcription) pathway. LIF acts by binding to heterodimers of LIF receptor (LIFR) and gp130. The LIF gene refers encodes a 202 amino acid precursor which is processed to yield a 180 amino acid biologically protein provided in the GenPept database under accession number NP_002300 and which is encoded by the nucleic acid sequence provided in the GenBank database under accession number NM 002309.

As used herein, the term “LIF antagonist” to a molecule (natural or synthetic) that blocks signal transduction by LIF and inhibit the biological activity of LIF. Specific examples of LIF antagonists include molecules that bind to LIF, molecules that inhibit LIF expression, molecules that bind to an LIF receptor, molecules that inhibit the expression of an LIF receptor, molecules that bind to GP130, and molecules that inhibit GP130 expression.

For instance, one class of antagonists will bind to LIF with sufficient affinity and specificity to neutralize LIF such that it has no effect on its receptors. Included within this group of antagonists are antibodies. Another class of antagonists are molecules based on an interaction between the LIF its receptors (LIFR and/or GP130). Such antagonists include fragments of the LIF receptor or small bioorganic molecules, e.g. peptidomimetics, that prevent the interaction between LIF and its receptors (LIFR and/or GP130).

In one embodiment, the LIF antagonist is an inhibitor of LIF gene expression. In a particular embodiment, the inhibitor of LIFR gene expression is selected form the group consisting of an antisense oligonucleotide, a small inhibitory RNA (siRNA) and a ribozyme

In another embodiment, the LIF antagonist is an anti-LIF antibody.

Several neutralizing antibodies specific to LIF are described in U.S. Pat. No. 5,654,157; U.S. Pat. No. 5,654,157; U.S. Pat. No. 5,573,762; U.S. Pat. No. 5,837,241 and US2013142808.

As used herein, the term “LIF receptor” (LIFR) also known as CD118 refers to a subunit of a receptor for leukemia inhibitory factor. LIFR gene refers encodes a 1097 amino acid polypeptide provided in the GenPept database under accession number NP_001121143 and which is encoded by the nucleic acid sequence provided in the GenBank database under accession number NM_001127671.

In one embodiment, the LIFR antagonist is an inhibitor of LIFR gene expression. In a particular embodiment, the inhibitor of LIFR gene expression is selected form the group consisting of an antisense oligonucleotide, a small inhibitory RNA (siRNA) and a ribozyme.

In another embodiment, the LIF antagonist is an anti-LIFR antibody.

As used herein, the term “Glycoprotein 130” (GP130), also known as IL6-ST or CD130, is well known in the art and refers to a transmembrane protein which is the founding member of the class of all cytokine receptors. It forms one subunit of type I cytokine receptors within the IL-6 receptor family. GP130 gene refers encodes a 857 amino acid polypeptide provided in the GenPept database under accession number NP_001177910 and which is encoded by the nucleic acid sequence provided in the GenBank database under accession number NM_001190981.

In one embodiment, the LIFR antagonist is an inhibitor of GP130 gene expression. In a particular embodiment, the inhibitor of GP130 gene expression is selected form the group consisting of an antisense oligonucleotide, a small inhibitory RNA (siRNA) and a ribozyme. Accordingly, approaches for inhibiting GP130 expression has been disclosed in the International Patent application No. WO2011/127175.

In one particular embodiment, the sequence of the siRNA targeting GP130 is CUAAUUACAUUGUCUGGAA represented by SEQ ID NO: 5.

In one particular embodiment, the sequence of the siRNA targeting GP130 is CUAAGGAGCAAUAUACUAU represented by SEQ ID NO: 6.

In another embodiment, the LIF antagonist is an anti-glycoprotein 130 (GP130) antibody.

Through paracrine signaling molecules and more particularly through TGFβ1 and LIF, cancer cells activate stromal fibroblasts, which in turn, affect the proliferation, invasion and migration of the cancer cells. Thus, activated fibroblasts such as carcinoma-associated fibroblasts promote malignant and invasive behaviour. It should be noted that massive growth of solid cancer such as epithelial cell cancer is associated with a number of characteristic cellular and molecular changes in the surrounding stroma cells.

Activation of fibroblasts in cancer stroma is a well-known mechanism by which quiescent fibroblasts are activated in tumor stroma and become cancer-associated fibroblasts (CAF). CAF then acts as key regulators of the paracrine signaling between stromal and cancer cells. Such mechanism of activation of fibroblasts is described in Rasanen et al., 2010.

In another aspect, the invention thus relates to a LIF antagonist for use in inhibiting the pro-invasive stromal fibroblast activation in a patient in need thereof.

In one embodiment, the patient in need thereof is a patient affected with a Leukemia Inhibitory Factor (LIF)-expressing solid cancer.

In one embodiment, the LIF antagonist inhibits the pro-invasive stromal fibroblast activation of SMA expressing fibroblasts (α-SMA+ fibroblasts). Such activated α-SMA+ fibroblasts are known as to carcinoma-associated fibroblasts (CAFs).

In one embodiment, the inhibitor of the LIF antagonist inhibits the pro-invasive stromal fibroblast activation of SMA expressing fibroblasts (α-SMA-fibroblasts).

The invention also relates to a method for inhibiting the pro-invasive stromal fibroblast activation in a patient in need thereof, comprising a step of administering to said patient a therapeutically effective amount of a LIF antagonist.

In one embodiment, the patient in need thereof is a patient affected with a Leukemia Inhibitory Factor (LIF)-expressing solid cancer.

As used herein, the terms “stroma” or “microenvironment” refer to the connective, supportive framework of a biological cell, tissue, or organ. As used herein, the term “tumor microenvironment” refers to the cellular environment in which the tumor exists, including the area immediately surrounding fibroblasts, leukocytes and endothelial cells and the extracellular matrix (ECM). The term “stromal cells” include fibroblasts, leukocytes and vascular cells. Accordingly, cells of a tumor microenvironment comprise malignant cells in association with non-malignant cells that support their growth and survival. The non-malignant cells, also called stromal cells, occupy or accumulate in the same cellular space as malignant cells, or the cellular space adjacent or proximal to malignant cells, which modulate tumor cell growth or survival. Non-malignant cells of the tumor microenvironment include fibroblasts, epithelial cells, vascular cells (including blood and lymphatic vascular endothelial cells and pericytes), resident and/or recruited inflammatory and immune (e.g., macrophages, dendritic cells, granulocytes, lymphocytes, etc.). These cells and especially activated fibroblasts actively participate in metastasis development.

The term “therapeutically effective amount” is intended to mean that amount of a drug that will elicit the biological or medical response of a patient that is being sought by a clinician. Pharmaceutical compositions may be administered in any conventional dosage formulation. Pharmaceutical compositions typically comprise at least one active ingredient, as defined above, together with one or more pharmaceutical acceptable excipient.

In still another aspect, the invention further relates to an inhibitor of the JAK1/STAT3 signalling pathway for use in inhibiting pro-invasive stromal fibroblast activity in a patient in need thereof.

In one embodiment, the patient in need thereof is a patient affected with a Leukemia Inhibitory Factor (LIF)-expressing solid cancer.

In one embodiment, the inhibitor of the JAK1/STAT3 signalling pathway inhibits the pro-invasive stromal fibroblast activity of SMA expressing fibroblasts (α-SMA+ fibroblasts). Such activated α-SMA+ fibroblasts are known as to carcinoma-associated fibroblasts (CAFs).

In another embodiment, the inhibitor of the invention inhibits the pro-invasive stromal fibroblast activity of non-SMA expressing fibroblasts (α-SMA-fibroblasts). Such activated α-SMA-fibroblasts also display a pro-invasive activity as disclosed herein.

The invention further relates to a method for inhibiting the pro-invasive stromal fibroblast activity in a patient in need thereof, comprising a step of administering to said patient a therapeutically effective amount of an inhibitor of the JAK1/STAT3 signalling pathway.

In one embodiment, the patient in need thereof is a patient affected with a Leukemia Inhibitory Factor (LIF)-expressing solid cancer.

In another aspect, the invention relates to an inhibitor of the JAK1/STAT3 signalling pathway for use in inhibiting pro-tumorigenic ECM remodeling and/or track formation and/or collective carcinoma cell invasion in a patient in need thereof.

In one embodiment, the patient in need thereof is a patient affected with a Leukemia Inhibitory Factor (LIF)-expressing solid cancer.

The invention also relates to a method for inhibiting pro-tumorigenic ECM remodeling and/or track formation and/or collective carcinoma cell invasion in a patient in need thereof, comprising a step of administering to said patient a therapeutically effective amount of an inhibitor of the JAK1/STAT3 signalling pathway.

In one embodiment, the patient in need thereof is a patient affected with a Leukemia Inhibitory Factor (LIF)-expressing solid cancer.

As used herein, the term “extracellular matrix” (ECM) refers to a complex mixture of macromolecules that accumulates within tissues in close apposition to cell surfaces. ECM contains secreted macromolecules such as collagens I, III and IV; fibronectin; laminins; and various proteoglycans. These macromolecules can be organized to provide cohesion to the tissue and can contribute to its structural and mechanical properties. ECM can act as a depository for, and release site of, potent secreted growth factors, and is known to influence growth, survival and differentiation of the cells it surrounds. It has long been known that tumor-derived ECM is biochemically distinct in its composition compared with normal ECM.

As used herein, the term “ECM remodelling” refers to a mechanism crucial for tumour malignancy and metastatic progression, which ultimately cause over 90% of deaths from cancer. Indeed, changes in the tumor microenvironment through remodeling of the ECM are important for metastatic dissemination. Specifically, the breakdown of normal ECM and its replacement with tumor ECM in the microenvironment leads to altered physiological cues that act as key drivers for malignant progression. Such ECM remodelling is characterized by increased activated fibroblasts, collagen deposition and assembly of collagen fibers as well as crosslinking of said collagen fibers in order to increase the overall stiffness of stromal environment providing the mechanical force necessary for tumor cell migration and invasion.

The microenvironment is important for tumor invasion and metastasis since cancer cells migrate along tracks made of extracellular matrix (ECM) collagen fibers. Activated fibroblasts facilitate tumor cell invasion through protease- and force-dependent generation of ECM tracks¹⁸. Carcinoma-associated fibroblasts (CAFs), the main cell component of the desmoplastic stroma of solid cancers, thus deposit ECM and remodel the tumor stroma.

### Pharmaceutical Compositions of the Invention

Any therapeutic agent of the invention as above described may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

To prepare pharmaceutical compositions, an effective amount of therapeutic agent of the invention according to the invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions of the therapeutic agents as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The therapeutic agents of the invention can be formulated into a composition in a neutral or salt form. Pharmaceutically acceptable salts include the acid addition salts (and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, (e.g. aluminium monostearate and gelatine).

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the therapeutic agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. In addition to the compounds formulated for parenteral administration, such as intravenous or intramuscular injection, other pharmaceutically acceptable forms include, e.g. tablets or other solids for oral administration; time release capsules; and any other form currently used.

In one aspect, the invention relates to a pharmaceutical composition comprising an inhibitor of the Janus Kinase 1 (JAK1)/Signal Transducer and Activator of Transcription 3 (STAT3) signalling pathway for use in preventing metastasis in a patient in need thereof.

In another aspect, the invention relates to a pharmaceutical composition comprising an inhibitor of the JAK1/STAT3 signalling pathway for use in inhibiting the pro-invasive stromal fibroblast activity in a patient in need thereof.

In still another aspect, the invention relates to a pharmaceutical composition comprising an inhibitor of the JAK1/STAT3 signalling pathway for use in inhibiting pro-tumorigenic ECM remodeling and/or track formation and/or collective carcinoma cell invasion in a patient in need thereof.

In one embodiment, the patient in need thereof is a patient affected with a Leukemia Inhibitory Factor (LIF)-expressing solid cancer.

In a particular embodiment, the inhibitor is 3-Cyclopentyl-3-[4-(7H-pyrrolo[2,3-d]pyrimidin-4-yl)-1H-pyrazol-1-yl]propanenitrile (ruxolitinib) or a pharmaceutically acceptable salt thereof.

In one aspect, the invention relates to a pharmaceutical composition comprising a Leukemia Inhibitory Factor (LIF) antagonist for use in preventing metastasis in a patient in need thereof.

In another aspect, the invention relates to a pharmaceutical composition comprising a LIF antagonist for use in inhibiting the pro-invasive stromal fibroblast activation in a patient in need thereof.

In a particular embodiment, the LIF antagonist is an anti-LIF antibody.

In one embodiment, the LIF antagonist inhibits the pro-invasive stromal fibroblast activation of SMA expressing fibroblasts (α-SMA+ fibroblasts or CAFs.

In one embodiment, the inhibitor of the LIF antagonist inhibits the pro-invasive stromal fibroblast activation of SMA expressing fibroblasts (α-SMA-fibroblasts).

### Combination Therapies of the Invention

In one aspect, the invention relates to a pharmaceutical composition comprising an inhibitor of the JAK1/STAT3 signalling pathway according to the invention and a LIF antagonist according to the invention.

In still another aspect, the invention relates to a kit-of-part composition comprising an inhibitor of the JAK1/STAT3 signalling pathway according to the invention and a LIF antagonist according to the invention.

In a preferred embodiment, the JAK1/STAT3 signalling pathway is Ruxolitinib.

Pharmaceutical compositions or kit-of-part compositions of the invention may comprise an additional therapeutic agent.

In one embodiment, said additional therapeutic active agent is a chemotherapeutic agent. Non-limiting examples of chemotherapeutic compounds which can be used in combination treatments of the invention include, for example, conventional chemotherapeutic, radiotherapeutic and anti-angiogenic agents.

In one particular embodiment, said chemotherapeutic agent is a tyrosine kinase inhibitor (TKI).

A number of other TKIs are in late and early stage development for treatment of various types of cancer. Exemplary TKIs include, but are not limited to: BAY 43-9006 (sorafenib, Nexavar®) and SU11248 (sunitinib, Sutent®), Imatinib mesylate (Gleevec®, Novartis); Gefitinib (Iressa®, AstraZeneca); Erlotinib hydrochloride (Tarceva®, Genentech); Vandetanib (Zactima®, AstraZeneca), Tipifamib (Zarnestra®, Janssen-Cilag); Dasatinib (Sprycel®, Bristol Myers Squibb); Lonafamib (Sarasar®, Schering Plough); Vatalanib succinate (Novartis, Schering AG); Lapatinib (Tykerb®, GlaxoSmithKline); Nilotinib (Novartis); Lestaurtinib (Cephalon); Pazopanib hydrochloride (GlaxoSmithKline); Axitinib (Pfizer); Canertinib dihydrochloride (Pfizer); Pelitinib (National Cancer Institute, Wyeth); Tandutinib (Millennium); Bosutinib (Wyeth); Semaxanib (Sugen, Taiho); AZD-2171 (AstraZeneca); VX-680 (Merck, Vertex); EXEL-0999 (Exelixis); ARRY-142886 (Array BioPharma, AstraZeneca); PD-0325901 (Pfizer); AMG-706 (Amgen); BIBF-1120 (Boehringer Ingelheim); SU-6668 (Taiho); CP-547632 (OSI); (AEE-788 (Novartis); BMS-582664 (Bristol-Myers Squibb); JNK-401 (Celgene); R-788 (Rigel); AZD-1152 HQPA (AstraZeneca); NM-3 (Genzyme Oncology); CP-868596 (Pfizer); BMS-599626 (Bristol-Myers Squibb); PTC-299 (PTC Therapeutics); ABT-869 (Abbott); EXEL-2880 (Exelixis); AG-024322 (Pfizer); XL-820 (Exelixis); OSI-930 (OSI); XL-184 (Exelixis); KRN-951 (Kirin Brewery); CP-724714 (OSI); E-7080 (Eisai); HKI-272 (Wyeth); CHIR-258 (Chiron); ZK-304709 (Schering AG); EXEL-7647 (Exelixis); BAY-57-9352 (Bayer); BIBW-2992 (Boehringer Ingelheim); AV-412 (AVEO); YN-968D1 (Advenchen Laboratories); Staurosporin, Midostaurin (PKC412, Novartis); Perifosine (AEterna Zentaris, Keryx, National Cancer Institute); AG-024322 (Pfizer); AZD-1152 (AstraZeneca); ON-01910Na (Onconova); and AZD-0530 (AstraZeneca).

Chemotherapeutic agents have different modes of actions, for example, by influencing either DNA or RNA and interfering with cell cycle replication. Examples of chemotherapeutic agents that act at the DNA level or on the RNA level are anti-metabolites (such as 5-Azacytidine, Azathioprine, Cytarabine, Fludarabine phosphate, Fludarabine, Gemcitabine, cytarabine, Cladribine, capecitabine 6-mercaptopurine, 6-thioguanine, methotrexate, 5-fluoroouracil and hyroxyurea; alkylating agents (such as Melphalan, Busulfan, Cis-platin, Carboplatin, Cyclophosphamide, Ifosphamide, Dacarabazine, Procarbazine, Chlorambucil, Thiotepa, Lomustine, Temozolamide); anti-mitotic agents (such as Vinorelbine, Vincristine, Vinblastine, Docetaxel, Paclitaxel); topoisomerase inhibitors (such as Doxorubincin, Amsacrine, Irinotecan, Daunorubicin, Epirubicin, Mitomycin, Mitoxantrone, Idarubicin, Teniposide, Etoposide, Topotecan); antibiotics (such as actinomycin and bleomycin); asparaginase; anthracyclines or taxanes.

Additional chemotherapeutic agent may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Exemplary biologics drugs include, but are not limited to: anti-angiogenic agents such as Bevacuzimab (mAb, inhibiting VEGF-A, Genentech); IMC-1121B (mAb, inhibiting VEGFR-2, ImClone Systems); CDP-791 (Pegylated DiFab,VEGFR-2, Celltech); 2C3 (mAb, VEGF-A, Peregrine Pharmaceuticals); VEGF-trap (soluble hybrid receptor VEGF-A, P1GF (placenta growth factor) Aventis/Regeneron).

Accordingly, in one aspect, the invention relates to a pharmaceutical composition comprising an inhibitor of the JAK1/STAT3 signalling pathway and/or a LIF antagonist according to the invention and a chemotherapeutic agent.

In one embodiment, the chemotherapeutic agent is a DNA methyltransferase (DNMT) inhibitor.

In one embodiment, the pharmaceutical composition thus comprises an inhibitor of the JAK1/STAT3 signalling pathway, a LIF antagonist and a DNMT inhibitor.

Exemplary inhibitors include 5-azacytidine (Azacitidine), 5-aza-2′-deoxycytidine (decitabine), fazarabine, DHAC, Ara-C, zebularine, (−)-epigallocatechin-3-gallate, MG98, RG108 and the like. The structures of some of these compounds are shown below.

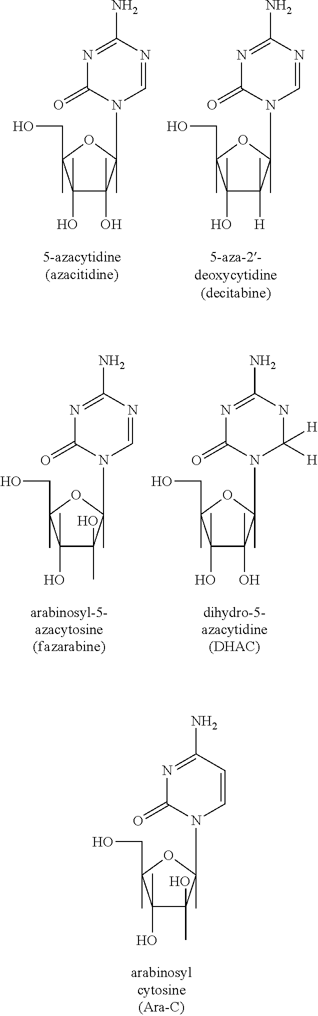

In a preferred embodiment, the DNMT inhibitor is Azacitidine.

In another aspect, the invention relates to a kit-of-part composition comprising an inhibitor of the JAK1/STAT3 signalling pathway and/or a LIF antagonist according to the invention and a chemotherapeutic agent.

In one embodiment, the chemotherapeutic agent is a DNMT inhibitor.

In one embodiment, the kit-of-part composition thus comprises an inhibitor of the JAK1/STAT3 signalling pathway, a LIF antagonist and a DNMT inhibitor.

In a preferred embodiment, the DNMT inhibitor is Azacitidine.

The terms “kit”, “product” or “combined preparation”, as used herein, define especially a “kit-of-parts” in the sense that the combination partners as defined above can be dosed independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes. When the administration is sequential, the first partner may be for instance administered 1, 2, 3, 4, 5, 6, 12, 18 or 24 h before the second partner.

In another aspect, the invention further relates to a pharmaceutical composition or a kit-of-parts composition comprising an inhibitor of the JAK1/STAT3 signalling pathway and a LIF antagonist for use in improving the survival time of a patient in need thereof.

In another aspect, the invention further relates to a pharmaceutical composition or a kit-of-parts composition comprising an inhibitor of the JAK1/STAT3 signalling pathway and and a DNMT inhibitor for use in improving the survival time of a patient in need thereof.

In a preferred embodiment, the JAK1/STAT3 signalling pathway is Ruxolitinib and the DNMT inhibitor is Azacitidine.

In one embodiment, the patient in need thereof is a patient affected with a Leukemia Inhibitory Factor (LIF)-expressing solid cancer.

In one embodiment, the survival time is progression-free survival (PFS).

The term “Progression-Free Survival” (PFS) in the context of the invention refers to the length of time during and after treatment during which, according to the assessment of the treating physician or investigator, the patient's disease does not become worse, i.e., does not progress. As the skilled person will appreciate, a patient's progression-free survival is improved or enhanced if the patient experiences a longer length of time during which the disease does not progress as compared to the average or mean progression free survival time of a control group of similarly situated patients.

In one embodiment, the survival time is Overall Survival (OS).

The term “Overall Survival” (OS) in the context of the invention refers to the average survival of the patient within a patient group. As the skilled person will appreciate, a patient's overall survival is improved or enhanced, if the patient belongs to a subgroup of patients that has a statistically significant longer mean survival time as compared to another subgroup of patients. Improved overall survival may be evident in one or more subgroups of patients but not apparent when the patient population is analysed as a whole.

### Diagnostic Methods of the Invention

In another aspect, the invention relates to an in vitro method for determining whether a patient is likely to benefit from a therapy with an inhibitor of the JAK1/STAT3 signalling pathway and/or a LIF antagonist, comprising a step of determining the expression level of LIF and/or a step of determining the level of phospho-Tyr705-STAT3 (PY-STAT3) in a biological sample obtained from said patient.

As used herein, the term “biological sample” has its general meaning in the art and refers to any sample which may be obtained from a patient. Examples of such samples include fluids, tissues, cell samples, organs, biopsies, etc. A preferred sample is a biological sample containing cancer cells and/or stromal cells (e.g. fibroblasts) such as a tumor biopsy sample.

In one embodiment, the patient has been previously diagnosed with a solid cancer (e.g. epithelial cancer or melanoma) or with a recessive dystrophic epidermolysis bullosa (RDEB).

In particular embodiment, the method for determining whether a patient is likely to benefit from a therapy with an inhibitor of the JAK1/STAT3 signalling pathway and/or a LIF antagonist comprises the steps of (i) determining the expression level of the LIF gene in a biological sample obtained from said patient, (ii) comparing said expression level with a predetermined reference value, wherein a increase in expression level of the LIF gene is indicative of metastasis or of a risk of metastasis, and (iii) subsequently administering to the patient in need thereof a therapeutically effective amount of an inhibitor of the JAK1/STAT3 signalling pathway and/or a LIF antagonist.

At step ii) of the method, the value for the expression level of LIF gene which is obtained at the end of step i) is compared with a predetermined reference value for the expression level of LIF. As used herein, the term “predetermined reference value” refers to the amount of LIF in biological samples obtained from the general population or from a selected population of subjects. The predetermined reference value can be a threshold value or a range. For example, the selected population may be comprised of apparently healthy subjects, such as individuals who have not previously had any sign or symptoms indicating the presence of cancer and/or metastasis.

In particular embodiment, the method for determining whether a patient is likely to benefit from a therapy with an inhibitor of the JAK1/STAT3 signalling pathway and/or a LIF antagonist comprises the steps of (i) determining the level of phospho-PY-STAT3 in a biological sample obtained from said patient, (ii) comparing said expression level with a predetermined reference value, wherein a increase in the level of PY-STAT3 is indicative of metastasis or of a risk of metastasis, and (iii) subsequently administering to the patient in need thereof a therapeutically effective amount of an inhibitor of the JAK1/STAT3 signalling pathway and/or a LIF antagonist.

At step ii) of the method, the value for the level of phospho-Tyr705-STAT3 (PY-STAT3) which is obtained at the end of step i) is compared with a predetermined reference value for the level of PY-STAT3. As used herein, the term “predetermined reference value” refers to the amount of PY-STAT3 in biological samples obtained from the general population or from a selected population of subjects. The predetermined reference value can be a threshold value or a range. For example, the selected population may be comprised of apparently healthy subjects, such as individuals who have not previously had any sign or symptoms indicating the presence of cancer and/or metastasis.

Typically, the level of PY-STAT3 is carried out by immunohistochemistry in a biopsy sample from the patient as described in the section Examples.

### Methods for Determining the Expression Level of the Biomarker of the Invention

Determination of the expression level of LIF gene may be performed by a variety of techniques. Generally, the expression level as determined is a relative expression level. For example, the determination comprises contacting the biological sample with selective reagents such as probes, primers or ligands, and thereby detecting the presence, or measuring the amount, of polypeptide or nucleic acids of interest originally in said biological sample. Contacting may be performed in any suitable device, such as a plate, microtiter dish, test tube, well, glass, column, and so forth. In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the biological sample.

As used herein, the term “determining” includes qualitative and/or quantitative detection (i.e. detecting and/or measuring the expression level) with or without reference to a control or a predetermined value. As used herein, “detecting” means determining if LIF is present or not in a biological sample and “measuring” means determining the amount of LIF in a biological sample. Typically the expression level may be determined for example by RT-PCR or immunohistochemistry (IHC) performed on a biological sample.

In a particular embodiment, the expression level may be determined by determining the quantity of mRNA.

Methods for determining the quantity of mRNA are well known in the art. For example the nucleic acid contained in the biological samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical.

In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above detection, staging, screening and monitoring methods may be assembled as a kit. Such a kit includes consensus primers and molecular probes. A particular kit also includes the components necessary to determine if amplification has occurred. The kit may also include, for example, PCR buffers and enzymes; positive control sequences, reaction control primers; and instructions for amplifying and detecting the specific sequences.

Accordingly, the invention also relates to a kit for performing a method above-mentioned, wherein said kit comprises means for determining the expression level of the LIF gene in a biological sample obtained from said patient.

In a particular embodiment, the methods of the invention comprise the steps of providing total RNAs extracted from a biological sample such a biological sample and subjecting the RNAs to amplification and hybridization to specific probes, more particularly by means of a quantitative or semi-quantitative RT-PCR.

In another particular embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g., Hoheisel, Nature Reviews, Genetics, 2006, 7:200-210).

In this context, the invention further provides a DNA chip comprising a solid support which carries nucleic acids that are specific to the LIF gene.

In a particular embodiment, the expression level of LIF may be determined by determining the quantity of protein encoded by LIF gene.

Such methods comprise contacting the biological sample with a binding partner capable of selectively interacting with the biomarker protein of interest present in the biological sample. The binding partner is generally an antibody that may be polyclonal or monoclonal, preferably monoclonal. Polyclonal antibodies directed against LIF are well known from the skilled man in the art such as polyclonal antibodies sc-1336 purchased from Santa Cruz Biotechnology (Santa Cruz, Calif.).

The antibodies of the invention may be monoclonal or polyclonal antibodies, single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies.

Whereas polyclonal antibodies may be used, monoclonal antibodies are preferred for they are more reproducible in the long run.

Procedures for raising “polyclonal antibodies” are also well known. Polyclonal antibodies can be obtained from serum of an animal immunized against the appropriate antigen, which may be produced by genetic engineering for example according to standard methods well-known by one skilled in the art. Typically, such antibodies can be raised by administering LIF subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. This and other procedures for raising polyclonal antibodies are disclosed by Harlow et al. (1988).

A “monoclonal antibody” refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988). Monoclonal antibodies (mAbs) may be prepared by administering LIF into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993).

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

The presence of the protein of interest may be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Labels are known in the art that generally provide (either directly or indirectly) a signal.

More particularly, an ELISA method may be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Alternatively, an immunohistochemistry (IHC) method may be used. IHC specifically provides a method of detecting a target protein in a biological sample or tissue specimen in situ. The overall cellular integrity of the sample is maintained in IHC, thus allowing detection of both the presence and location of the target of interest. Typically a biological sample is fixed with formalin, embedded in paraffin and cut into sections for staining and subsequent inspection by light microscopy. Current methods of IHC use either direct labelling or secondary antibody-based or hapten-based labelling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

Typically, LIF immunohistological staining may be carried out as described below in the Section Examples.

As previously described, probe, primers, aptamers or antibodies of the invention may be labelled with a detectable molecule or substance, such as a fluorescent molecule, a radioactive molecule or any others labels known in the art. Labels are known in the art that generally provide (either directly or indirectly) a signal.

The term “labelled”, with regard to the probe, primers, aptamers or antibodies of the invention, is intended to encompass direct labelling of the probe, primers, aptamers or antibodies of the invention by coupling (i.e., physically linking) a detectable substance to the probe, primers, aptamers or antibodies of the invention, as well as indirect labelling of the probe, primers, aptamers or antibodies of the invention by reactivity with another reagent that is directly labelled. Other examples of detectable substances include but are not limited to radioactive agents or a fluorophore (e.g. fluorescein isothiocyanate (FITC) or phycoerythrin (PE) or Indocyanine (Cy5)). Examples of indirect labeling include detection of a primary antibody using a fluorescently labelled secondary antibody and end-labelling of a DNA probe with biotin such that it can be detected with fluorescently labelled streptavidin. An antibody or aptamer of the invention may be labelled with a radioactive molecule by any method known in the art. For example radioactive molecules include but are not limited radioactive atom for scintigraphic studies such as I¹²³, I¹²⁴, In¹¹, Re¹⁸⁶, Re¹⁸⁸.

In one embodiment, the method comprises a further step of determining the collective cancer cell pattern of invasion. Immunohistochemical analyses of cancer invasion and cells junctions may also be carried out in order to determine the morphological pattern of invasion as described in Friedl et al., 2012.

In one embodiment, the method comprises a further step of determining the fibrotic microenvironment.

Collagen fibres are indicative of a dense ECM and correlates with the invasive potential of tumour cells. Assessment of collagen production and collagen fibres assembly in biopsy tumor sample by Sirius Red staining. Typically, collagen Sirius red staining may be carried out as described below in the Section Examples.

As discussed herein, the tumor microenvironment may display a fibrotic ECM characterized by altered biochemical and biomechanical properties. Accordingly, the term “fibrosis” is used synonymously with “fibroblast accumulation and collagen deposition”. Fibroblasts are connective tissue cells, which are dispersed in connective tissue throughout the body. Fibroblasts secrete a nonrigid extracellular matrix containing type I and/or type III collagen. In response to an injury to a tissue, nearby fibroblasts migrate into the wound, proliferate, and produce large amounts of collagenous extracellular matrix. Collagen is a fibrous protein rich in glycine and proline that is a major component of the extracellular matrix and connective tissue, cartilage, and bone. Collagen molecules are triple-stranded helical structures called α-chains, which are wound around each other in a ropelike helix. Collagen exists in several forms or types; of these, type I, the most common, is found in skin, tendon, and bone; and type III is found in skin, blood vessels, and internal organs.

The invention will be further illustrated by the following figures and examples. However, these examples and figures should not be interpreted in any way as limiting the scope of the present invention.

### FIGURES

FIG. 1: TGFβ1-signalling initiates and JAK-signalling sustains pro-invasive fibroblast property. A) Quantification of matrix contraction by hDF stimulated by TGFβ1 (2 ng·ml-1) for 7 days and subsequently treated by TGFβr-I inhibitor (SB431542) and pan-JAK inhibitor (P6) (n=3 in triplicates; mean+SD; ***p<0.001). Bottom panel shows scanned images of the contracted gel. B) Quantification of SCC12 cell organotypic invasion assay index induced by TGFβ1-activated hDF for 7 days and subsequently treated with TGFβr-I inhibitor (SB431542) or JAK kinase inhibitor (P6) (n=3; mean+SD; ***p<0.001).

FIG. 2: LIF mediates TGFβ1-dependent pro-invasive fibroblast activation. A) Quantification of mRNA level relative to the control (Veh) of OSM, IL6, G-CSF, LIF, CNTF and CT1 one hour after TGFβ1-stimulation. (n=3 in duplicates; mean+SD; ***p<0.001). B) ELISA quantification of LIF secreted in TGFβ1-stimulated hDF culture media. (UD=Undetectable; n=3 in duplicates; mean+SD; ***p<0.001). C) Quantification of matrix remodelling by hDF stimulated by TGFβ1 or LIF in the presence or absence of LIF (αLIF) and IL6 (αIL6) blocking antibodies for 7 days. (n=3 in triplicates; mean+SD; ***p<0.001). Bottom panel shows scanned images of the contracted gel. D) Quantification of SCC12 cell organotypic invasion index induced by hDF stimulated by TGFβ1 or LIF in the presence of LIF and/or IL6 blocking antibodies (n=3; mean+SD, ***p<0.001).

FIG. 3: LIF mediates TGFβ1-dependent acto-myosin contractility independent of α-SMA expression. A) Quantification of gel contraction by hDF cells cultured in the presence of SCC12 or SCC13 conditioned media (CM) in control (black histograms) depleted for either TGFβ1,2,3 (αTGFβ; grey histograms) or for LIF (αLIF; white histogram) at 10 g/ml of blocking antibodies. (n=3 in triplicates; mean+SD. ***p<0.001 and **p<0.01). B) Quantification of SCC12 cell invasion index. (n=3; mean+SD; ***p<0.001). C) Histograms represent quantification of αSMA positive hDF 5 days after stimulation by tumour cell conditioned media (n=3; mean+SD; ***p<0.001). D) Quantification of gel contraction by hDF grown in the presence of control (veh.) or TGFβ1-stimulated CAL33 conditioned media (CM) in control (black histograms) depleted for LIF (αLIF; grey) or TGFβ1,2,3 (αTGFβ1,2,3; white) at 10 μg/ml of blocking antibodies. (n=3 in triplicates; mean+SD. ***p<0.001).

FIG. 4: LIF and JAK signalling mediate malignant tumour microenvironment in vivo. A) ELISA quantification of LIF secreted in mouse breast cancer cells culture media. (UD=Undetectable; n=3 in duplicates). Bottom panel shows the cell invasion and metastatic capacities (Y=yes and N=no). B) Quantification of tumour invasion. Distance was calculated using ImageJ software by the mean of 5 measurements from 18 pictures for each condition (mean+SD, p=0.000045). C) Quantification of thickness and length of collagen bundles (each dots represents one fibres. Total quantified fibres: 67NR=96, 410.4=680 and 410.4+Ruxo=390).

FIG. 5: LIF overexpression in human skin SCC tumours correlates with assembled collagen fibre organisation. A) Quantification of total pixel scored. Human skin (n=4) and SCC (n=17) from grade 1 and 2+SD (*p<0.05). B) Quantification of thickness and length of collagen bundles: human normal skin (n=4) and SCC (n=17) from grade 1 and 2+SD. **p<0.01. C) Schematic representation of LIF mediates pro-invasive tumour microenvironment: Presence of TGFβ-family cytokines within the tumour mass results in LIF production by both tumour cell and fibroblast. LIF mediates paracrine and autocrine activation of pro-invasive fibroblasts through activation of the GP130/JAK1/STAT3 signalling cascade in fibroblasts. LIF also mediates TGFβ-dependent acto-myosin contractility independent of αSMA expression, which results in ECM remodelling and fibronectin deposition. Deciphering the role of TGFβ signalling in tumourigenesis reveals a distinct but collaborative role for TGFβ and LIF cytokines. TGFβ drives phenotypic fibroblast conversion into CAF-like cells, while LIF supports formation of a pro-invasive tumour microenvironment.

FIG. 6: A) Quantification of matrix remodelling by human CAF cells isolated from head-and neck tumour (hHN-CAF), lung tumour (hLu-CAF) and breast tumour (hBr-CAF) in control (black) and in the presence of SB431542 inhibitor (grey) or P6 (white) (n=3 in triplicates; mean+SD; ***p<0.001). B) Quantification of matrix remodelling by hDF (hDF-1, -2 and -3) in control (black) or stimulated by TGFβ1 (grey) in the presence of P6 (white) (n=3 in triplicates; mean+SD; ***p<0.001). C) Quantification of SCC12 cell organotypic invasion assay index. (n=3; mean+SD; ***p<0.001).

### EXAMPLE

### LIF Mediates Proinvasive Activation of Stromal Fibroblasts in Cancer

Material & Methods

Cell Culture:

Human Primary Dermal Fibroblasts (hDF-1, -2 and -3) were cultured in DMEM supplemented with 10% FCS. CAFs were cultured in DMEM supplemented with 10% FCS and insulin-transferrin-selenium (#41400-045; Invitrogen, Carlsbad, Calif.). Oral squamous cell carcinoma (OSCC) cell lines (SCC12, SCC13, CAL25, CAL27, CAL33, CAL60, CAL166, Detroit 562) were cultured in FAD media¹⁸. Melanoma (Mel501, A375P, A375M2, Sccl2, WM35, WM278, WM793 and Mel1205), breast (MDA-MB-231, MDA-MB-468), lung (A549), and colon (LS174) cancer cell lines were cultured in DMEM supplemented with 10% FCS. Human Normal Keratinocytes (hNK) isolated from healthy neonatal foreskins were cultured on a feeder layer of lethally irradiated 373-J2 fibroblasts in FAD medium, as described before 56. Mouse dermal fibroblasts and carcinoma cells were cultured in DMEM supplemented with 10% FCS. Primary fibroblasts and primary CAFs were used at passages 3 to 8 for each in vitro experiment. All cells used in this study are listed in Supplementary Table 1.

SUPPLEMENTARY TABLE 1
List and designation of all primary cells and cell lines used for the study.
Cancer cell lines Designation

Human fibroblasts
hPDF1, 2 & 3 Human primary dermal fibroblasts, isolated from foreskins biopsies
hHN-CAF Human immortalized head and neck carcinoma associated fibroblasts¹⁸
hLu-CAF Human primary lung carcinoma associated fibroblasts (PC 60163A1; Asterand)
hBr1-CAF Human primary breast carcinoma associated fibroblasts (PC 87332A1; Asterand)
Human Primary Keratinocytes
hNK Human primary keratinocytes, isolated from foreskins biopsies
Human Squamous Cell Carcinoma cell lines
SCC12 Human epidermoid carcimoma (gift from E. Sahai)
SCC13 Human epidermoid carcinoma (gift from T. Magnaldo)
SCC25 Human carcinoma from tongue (ATCC number CRL-1628)
CAL27 Human carcinoma from tongue (ATCC number CRL-2095)
CAL33 Human head and neck SCC (gift from G. Milano)
CAL60 Human head and neck SCC (gift from G. milano)
CAL166 Human head and neck SCC (gift from G. Milano)
Detroit 562 Human carcinoma from pharynx (ATCC number CCL-138)
Colon Carcinoma cell line
LS174 human adenocarcinoma (ATCC number CL-188)
Lung Carcinoma cell line
A549 Human carcinoma from lung (ATCC number CCL-185)
Breast Carcinomas
MDA-MD-231 Human mammary adenocarcinoma (ATCC number HTB-26)
MDA-MD-468 Human mammary adenocarcinoma (ATCC number HTB-130)
Melanoma cell lines
A375P Human melanoma (parental) (gift from C. Marshall)
A375M2 Human melanoma (highly metastatic derived from A375P) (gift from C. Marshall)
Mel501 Human melanoma (gift from S. Tartare-Deckert)
Sbcl2 Human melanoma (gift from S. Tartare-Deckert)
WM35 Human melanoma (gift from S. Tartare-Deckert)
WM278 Human melanoma (gift from S. Tartare-Deckert)
WM793 Human melanoma (gift from S. Tartare-Deckert)
Mel1205 Human melanoma (gift from S. Tartare-Deckert)
Murine Fibroblast
mDF Murine primary dermal fibroblasts, isolated from back skin of Balb/C mice.
Murine Breast Carcinoma
67NR Breast tumour cell spontaneously arising from female Balb/C mice (Tumourigenic;
non-invasive)
4T07 Breast tumour cell spontaneously arising from female Balb/C mice (Tumourigenic;
invasive; non-metastatic)
410.4 Breast tumour cell spontaneously arising from female Balb/C mice (Tumourigenic;
invasive; metastatic)
4T1 Breast tumour cell spontaneously arising from female Balb/C mice (Tumourigenic;
invasive; metastatic)

Organotypic Invasion Assays:

In this assay, hDF or CAFs were cultured during one week in serum-free medium (with either vehicle or cytokines) or CM before embedding in matrix gel. 5×10⁵ fibroblasts were embedded in a 1 ml mixture of collagen I and Matrigel18. After the gel was set at 37° C. for 1 h, 5×10⁵ SCC12 were plated on top of the gel in complete media. The next day, the gel was lifted onto individual collagen coated nylon discs resting on metal bridges and fed from underneath with daily changes of complete media (with either vehicle or inhibitors), allowing the epithelial layer to grow at an air-liquid interface.

After 5 days, cultures were fixed (4% paraformaldehyde+0.25% glutaraldehyde in PBS) and processed for haematoxylin and eosin (H&E) staining by standard methods. Invasion assay was measuring the total area of SCC cells and the area of non-invading SCC cells using imageJ software (http://rsbweb.nih.gov/ij/). The value of invasion index shown is the average 1−(non-invading area/total area) of at least ten fields from three or more independent experiments. Error bars are the standard deviation.

Matrix Remodelling Assay:

2.5×10⁴ Fibroblasts were embedded in 100 μl of matrix gel (the Collagen-Matrigel mixture used for organotypic invasion assays) and plated into wells of a 96 well plate⁵⁷. After 1 h at 37° C., the matrices were overlaid with 100 μl serum-free medium (with either vehicle, cytokines or inhibitors) or CM. Every 2 days, medium was changed and at day 6 the gels were photographed and the respective diameters of the well and gel were measured using ImageJ. The percentage contraction was calculated using the formula 100*(well diameter−gel diameter)/well diameter.

Collagen Sirius Red Staining:

After embedding in paraffin, tissue sections from organotypic culture, skin from human breast and foreskin, and human skin carcinomas were prepared on slides using standard methods. Collagen assembly was evaluated by Sirius Red staining. Briefly, sections were first incubated with Weigert's iron hematoxylin (#C0231, Diapath, Martinengo, Italy) for 8 minutes and washed in distilled water for 10 minutes. Sections were next incubated with 1% Sirius Red in saturated picric acid for 1 hour. The slides were then washed twice with 0.5% acetic acid in distilled H2O, dehydrated and mounted using classical procedures. Sections were analysed using polarized light imaging and confocal laser microscopy with a LSM 5 EXCITER confocal microscope (Carl Zeiss, Germany). Collagen fibres thickness and length were quantified using ImageJ software.

Orthotopic Tumours in BALB/c Mice:

6-8 weeks old female BALB/c mice were anesthetized using ketamine and xelazine by peritoneal injection. Skin was incised and 5.10³ 67NR or 410.4 cells (5.10³) were injected into the right and left 4th mammary fat pad in 10 μl of PBS for each mouse. 67NR group consisted of 2 mice (4 primary tumours), 410.⁴ group consisted of 7 mice (14 tumours) and 410.4 with additional treatment of Ruxolitinib (30 mg/kg/day) group consisted of 5 mice (10 tumours). Mice were sacrificed 30 days post-injection and Ruxolitinib treatment started 7 days after injection, primary tumours were removed and fixed in PBS containing 3.7% formalin for 8 hours, followed by was with PBS and transfer to 70% ethanol, and then embedded in paraffin, sectioned and stained with haematoxylin and eosin. Immunohistochemistry detection using anti-LIF, anti-p-STAT3 and anti-alpha-SMA antibodies was performed on paraffin sections following manufactures instructions.

Neutralizing Antibody Methods:

Neutralizing antibody against LIF (AB-250-NA, R&D, Minneapolis, Minn.), IL-6 R (AB-227-NA, R&D) or TGF beta-1,2,3 (MAB1835, R&D) were used at 10 μg/ml otherwise stated. Neutralizing antibody were incubated one hour with conditioned media before being used for matrix remodelling assay and fibroblasts stimulation to neutralize the biological activity of selected cytokines.

ELISA:

For the quantitative determination of LIF protein levels secreted to the media, we used the human LIF ELISA Kit (#ELH-LIF-001, Raybiotech Inc, Norcross, Ga.) following manufacturer's specifications.

Conditioned Media Preparation:

Cancer Cells were grown to confluence, washed twice with PBS and then incubated in serum-free medium at 37° C. After 48 hours, conditioned medium was collected, centrifugated at 5000 g for 5 minutes to remove cell debris and the supernatant stored at −80° C. For immunobloting analysis, fibroblasts were stimulated with cancer cells conditioned media for 5 min, otherwise stated, and lysed as described in the immunbloting section. To analyse LIF secretion upon TGFβ-1 stimulation, fibroblasts were stimulated with 2 ng/ml TGF beta-1 for 1, 4, 24 or 48 h in serum-free medium and conditioned media was prepared as below for ELISA analysis.

SiRNA Transfections:

Fibroblasts were transfected using Dharmafect 3 (#T-2002-02; Dharmacon, inc., Lafayette, Colo.). Briefly, cells were plated at 60% confluence and subjected to transfection the following day using 100 nM final concentration of siRNA. SiRNA was purchased from Dharmacon and sequences are listed below. For Organotypic invasion assays, fibroblasts were transfected once at day 0 before being cultured in serum-free medium for one week with vehicle or cytokines, and a second time at day 7, the day before embedding in matrix gel.

| (SEQ ID NO: 1) | |
|---|---|
| JAK1 #2 | GACAUGAUAUUGAGAACGA |
| (SEQ ID NO: 2) | |
| JAK1 #3 | UUACAAGGAUGACGAAGGA |
| (SEQ ID NO: 3) | |
| STAT3 #1 | GAGAUUGACCAGCAGUAUA |
| (SEQ ID NO: 4) | |
| STAT3 #3 | CCAACAAUCCCAAGAAUGU |
| (SEQ ID NO: 5) | |
| GP130-IL6ST #1 | CUAAUUACAUUGUCUGGAA |
| (SEQ ID NO: 6) | |
| GP130-IL6ST #2 | CUAAGGAGCAAUAUACUAU |

ROCK-ER Fibroblasts:

pBABE puro ROCK-ER plamsid³⁰ was transfected into Phoenix retroviral packaging cells using calcium phosphate transfection method. After 8 hours, cells were rinsed with PBS and medium was changed. The following day, medium was replaced with 10% inactivated FCS supplemented medium and cells were cultured for 24 hours at 32° C. with 5% CO2. Supernatant was next collected and centrifugated at 1500 rpm for 5 min and filtered through a 0.45 micron filter. Exponentially growing fibroblasts were infected with undiluted retroviral supernatant mixed with 5 μg/ml Polybrene and selected with 5 μg/ml puromycin to establish stable cell culture.

Cytokines:

TGF beta-1 (#100-21, Peprotech, Rocky Hill, N.J.) was used at 2 ng·ml-1. Recombinant human LIF (#LIF1005. Millipore, Billerica, Mass.), was used at 1 ng·ml-1. Recombinant human IL-6 (#HZ-1019; Humanzyme) was used at 10 ng·ml-1.

Antibody and Inhibitors:

For western blot analysis, antibodies against STAT3 (#9134), pY705-STAT3 (#9145), SMAD2 (#3122), pSer465/467-SMAD2 (#3108), pY1022/1023-JAK1 (#3331), MLC2 (#3672), pThr18/19-MLC2 (#3674) were purchased from Cell Signaling (Cell Signaling Technology, Beverly Mass.), α-tubulin from sigma (T4026, Sigma, Saint Louis, Mo.); JAK1 from Biolegend (#604402, Biolegend, San Diego, Calif.), GP-130 (#sc-655) and RhoA (#sc-418, Santa Cruz Biotechnology, Santa Cruz, Calif.), and alpha-smooth muscle actin (α-SMA) from Abcam (#Ab5694, Abcam, Cambridge, Mass.). For immunofluorescence staining, antibodies against α-SMA and fibronectin were purchased from Sigma (#A2547 and F3648 respectively, Sigma, Saint Louis, Mo.). Chemical inhibitors used: Pyridone 6 (#42009, Calbiochem, Los Angeles, Calif.) was used at 5 M, SB431542 (#1614, Tocris bioscience, Ellisville, Mo.) at 10 μM, Y27632 (#1254, Tocris bioscience, Ellisville, Mo.) and Actinomycin D (#856258, Sigma, Saint Louis, USA) at 5 μg/ml.

Western Blot:

For immunoblotting analysis, cells were lysed on ice in lysis buffer (25 mM Tris (pH 6.8), 2% Sodium dodecyl sulfate (SDS), 5% glycerol, 1% beta-mercaptoethanol, 0.01% bromophenol blue). Equal amounts of protein from each sample were loaded on sodium dodecyl sulfate-polyacrylamide gel electrophoresis, separated, and transferred onto nitrocellulose. The immunoblots were blocked by incubation in 5% bovine serum albumin, 10 mM Tris-HCl (ph7.5), 500 mM NaCl, 0.1% Tween 20 for 30 min at room temperature, probed with specific antibodies and then with secondary antibodies using common classical methods. Immunodetection was performed using chemiluminescent HRP subtrate (#WBKLS0500, Millipore, Billerica, Mass.).

RT-qPCR Analysis:

RNA was isolated from total cell lysates using RNeasy Mini kit (#217004, Qiagen, Turnberry Ln Valencia, Calif.) according to the manufacturer's instructions. Reverse Transcription was performed using 500 ng cytoplasmic RNA using Superscript II reverse transcriptase (#18064-014, Invitrogen, Carlsbad, Calif.). Real time PCR was performed using Fast SYBR Green Master Mix (#18064-014; Applied Biosystems, Foster City, Calif.) in duplicates according to the recommendations of the manufacturer on an ABI Prism 7900HT sequence detection system (Applied Biosystems, Foster City, Calif.). Relative expression of the respective gene was determined after normalization to GAPDH and calculated with the following formula: relative expression level=2ddCT

LIF Immunohistological Staining:

Formalin-fixed tissues (3,7% in PBS) were transferred to 70% ethanol, embedded in paraffin wax and sectioned at 7 μm. After deparafination, microwave antigen retrieval was performed in Na-citrate buffer (10 mM, pH6; 5 min at 900 W and 25 min at 150 W) and sections washed three times in PBS (5 min per wash). Endogenous peroxidase activity was then blocked in 1% H2O2 in water for 10 min and sections were washed (3*5 min in PBS). After incubation in blocking buffer for two hours (10% rabbit serum (S-5000, Vector, Burlingame, Calif.); 0.3% Triton X100 in PBS) sections were incubated with LIF primary antibody (#sc-1336, Santa Cruz Biotechnology, Santa Cruz, Calif.) diluted 1:50 in blocking buffer overnight at 4° C. For negative controls, rabbit IgG replaced the primary antibody. After three washes in PBS (5 min per wash), sections were incubated with biotinylated anti-goat IgG (#BA-5000, Vector, Burlingame, Calif.) diluted 1:400 in PBS for 30 min and washed in PBS (3*5 min). Samples were then processed using Vectastain ABC kit (#PK4001, Vector, Burlingame, Calif.) and DAB perroxidase substrate kit (#SK4100, Vector, Burlingame, Calif.) according to manufacturer's instructions. Sections were next counterstained with hematoxylin for 5 sec, rinsed in water, blued 10 sec in 0.08% ammonia water, dehydrated, cleared, and mounted with cover clips.

Immunofluorescence Staining:

Cells were grown on glass coverslips, fixed in 3% paraformaldehyde for 20 min at room temperature, rinsed twice in PBS and in 50 mM NH4Cl for 10 min and permeabilized with 0.5% Triton X-100 for 5 min. Cells were next blocked in PBS containing 1% bovine serum albumin (BSA) for 30 min and incubated with anti-alpha smooth muscle actin (1/400), or anti-fibronectin (1/400) antibodies in blocking buffer overnight at 4° C. After two washes in PBS, cells were incubated in the presence of a secondary antibody conjugated to Alexa 488 (1/400) for 40 min, rinsed twice in PBS, stained with DAPI (2 ug/ml) for 5 min, rinsed in water and the coverslips mounted onto glass slides using mounting media.

Statistical Analysis:

Student's t test was performed for quantifications of invasion assay, matrix remodelling assay, ELISA test and qPCR results (***p<0.001; **p<0.01; *p<0.05).

Results

TGFβ1 Confers Pro-Invasive Properties to Fibroblasts Via a JAK1/STAT3-Dependent Signalling Pathway:

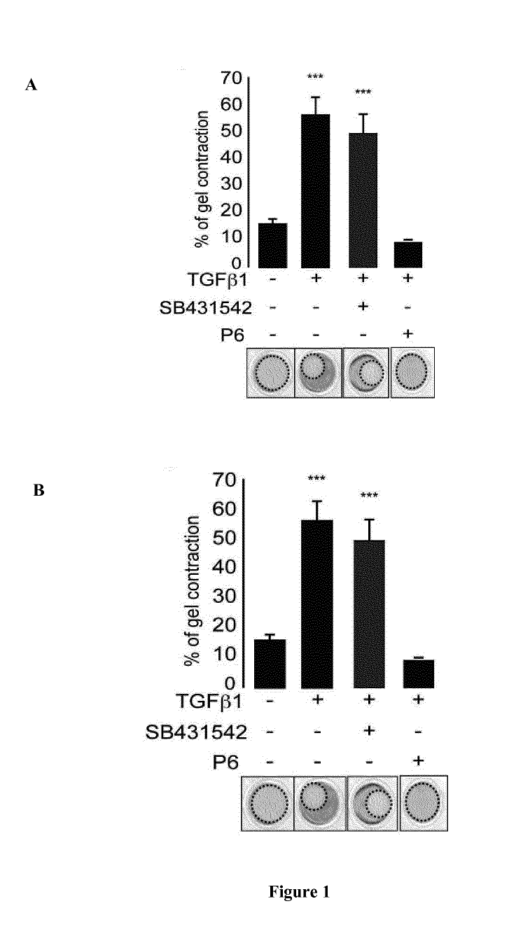

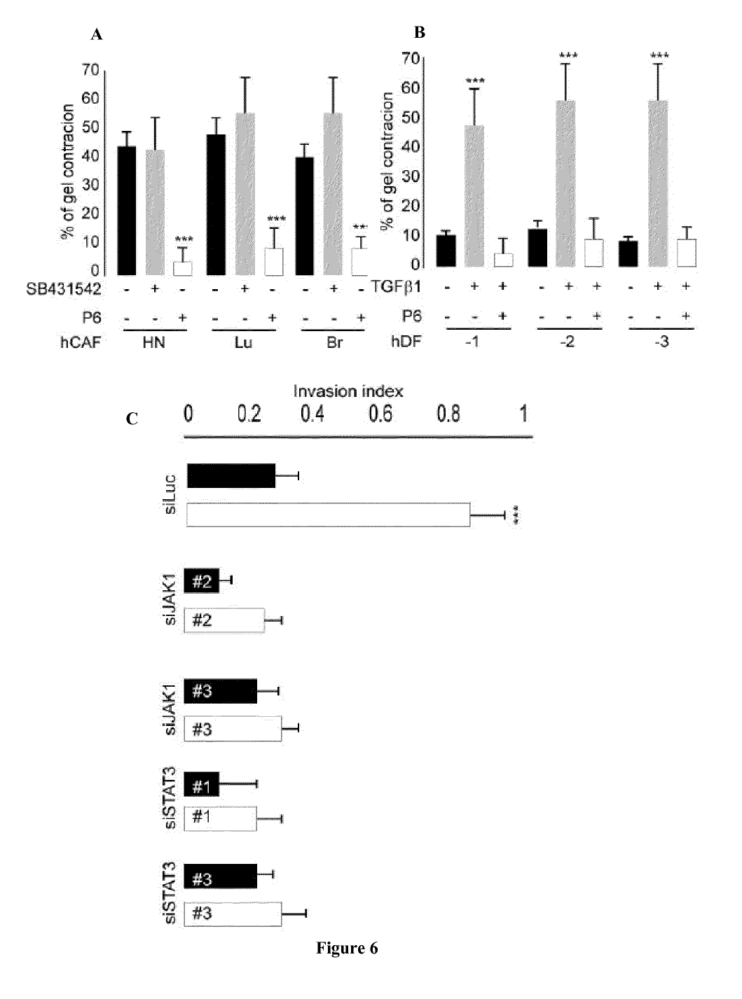

Human CAF isolated from head-and-neck squamous cell (hHN-CAF), lung (hLu-CAF) or breast (hBr-CAF) carcinoma (Supp. Table 1) support pro-invasive extracellular matrix (ECM) remodelling, as visualized in vitro by their capacity to contract collagen gels (FIG. 6A; black bars) and to promote human carcinoma SCC12 cell invasion, while human primary dermal fibroblasts (hDF) displayed no appreciable contractile phenotype and failed to induce SCC12 cell collective invasion. Because the TGFβ cytokines, including TGFβ1, 2 and 3, are known to promote myofibroblast activation during wound healing and tumourigenesis^{21,22}, we speculated that the TGFβ/SMAD signalling pathway might be responsible of the hDF conversion into contractile and pro-invasive hCAF-like cells. Indeed, in hDF, transient pulse of TGFβ1 stimulation induced both contractility (FIGS. 1A and 6B) and pro-invasive properties (FIG. 1B), and, in agreement with the notion that JAK kinase activity is needed for pro-invasive track formation by hHN-CAF²⁸, we found that hLu-CAF and hBr-CAF cells required JAK signalling to sustain their pro-invasive potential (FIG. 6A; P6 inhibitor) but not the TGFβr-I dependent signalling (Figures and 6A; SB431542 inhibitor), which indicated that TGFβ signalling might be dispensable for long-term maintenance of a hCAF pro-invasive phenotype. The possible contribution of the JAK kinase signalling to the pro-invasive properties of TGFβ1-activated hDF was thus assessed using specific inhibitors. Similar to hCAF cells, the TGFβ1-activated fibroblasts were found to rely on JAK but not on TGFβr-I signalling to promote matrix contraction (FIGS. 1A, 6A and 6B) and SCC 12-cell collective invasion (FIG. 1B). This confirmed that TGFβ signalling is sufficient to promote but not necessary to sustain the pro-invasive activity, a function that relies on JAK kinase signalling. Further, according with previous results²⁸, and similar to hHN-CAF, the TGFβ1-activated hDF resulted to rely on JAK1/STAT3 specific signalling to acquire ability for pro-invasion track formation within the ECM (FIG. 6C). Accordingly, hCAF cells expressing α-SMA marker displayed an enhanced endogenous activity of STAT3, which correlates with their endogenous levels of collagen gel contractility (FIGS. 6A and 6B; black histograms). Taken together, these data suggest that a transient stimulation of hDF by TGFβ1 is sufficient to induce the pro-invasive phenotype in hCAF, which is then sustained by a JAK1/STAT3-dependent signalling.

LIF Supports TGFβ1-Dependent Acto-Myosin Contractility Toward a Pro-Invasive Tumour Microenvironment:

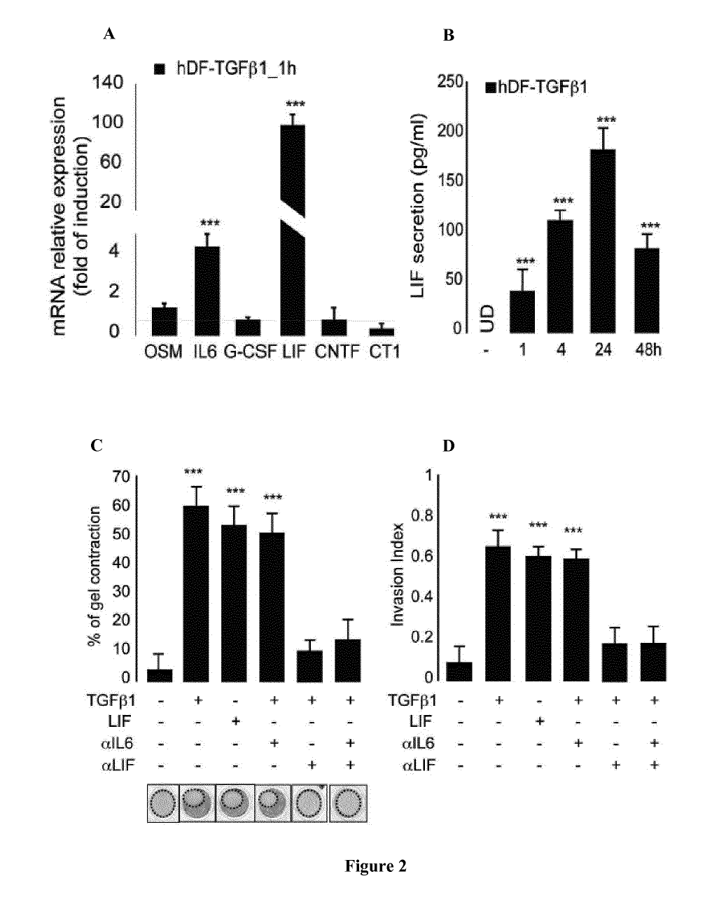

We next investigated the molecular mechanisms that govern TGFβ1-dependent JAK/STAT signalling activation. Stimulation of hDF by TGFβ1 induces phosphorylation of STAT3 and SMAD2 transcription factors. However, while SMAD2 activation occurs within 10 minutes, STAT3 activation is delayed up to one hour, which suggests involvement of distinct molecular mechanisms. Because IL6 cytokines are known to support JAK/STAT activation²⁹, transcription of the IL6 cytokine gene family members in TGFβ1-stimulated hDF was thus assessed by qRT-PCR, that disclosed a 100-fold increase of LIF and 5-fold increase of IL6 mRNA steady state levels (FIG. 2A). The respective role of these two cytokines was investigated using specific blocking antibodies, which identified LIF as the major cytokine mediating STAT3 phosphorylation upon TGFβ1 stimulation. Interestingly, LIF was detected in hDF cell culture medium 1 h after TGFβ1 stimulation, with a 24 h peak of 200 pg/ml (FIG. 2B), and, as expected, the pan-JAK inhibitor P6 blocked the STAT3 phosphorylation induced by TGFβ1. The involvement of LIF signalling in the TGFβ1-mediated STAT3 phosphorylation was confirmed by siRNA-mediated knock down of GP130-IL6ST. Indeed, silencing of the commune subunit receptor of the IL6 family cytokine GP130-IL6ST hampered STAT3 activation by TGFβ1 without affecting SMAD2 activation. These results support the conclusion that in hDF TGFβ1 relies on a LIF/GP130-IL6ST/JAK1 signalling cascade to induce STAT3 activation, and suggest that hDF just stimulated by LIF may promote onset of a pro-invasive microenvironment. According with this idea, both matrix remodelling (FIG. 2C) and SCC12 cell collective invasion were observed with hrLIF-stimulated hDF (FIG. 2D); further, LIF sequestration using a specific blocking anti-LIF antibody counteracted the action of TGFβ1 (FIGS. 2C-2D), In light of these observations, we deduced that TGFβ1 specifically activates the pro-invasive properties of hDF via the LIF/GP130-IL6ST/JAK1 signalling axis. Because JAK1 and ROCK cooperate to control acto-myosin contractility in hHN-CAF, which results in pro-invasive tracks formation within the ECM²⁸, the potential role of the RhoA/ROCK-dependent signalling pathway in LIF-mediated pro-invasive fibroblast activation was investigated. siRNA-mediated knock-down of RhoA expression, or pharmacological inhibition of Rho-kinase (ROCK) activity resulted in blockade of both TGFβ1 and LIF-dependent pro-invasive hDF activity. Long-term stimulation of both TGFβ and LIF cytokines also upregulated RhoA small GTPase and myosin light chain 2 (MLC2) proteins, leading to an increase in MLC2 phosphorylation at ser19, which attests for an increased activity. Finally, forced expression of an active form of ROCK (ROCK-ER)³⁰ following 4-hydroxytamoxifen (4OHT) treatment was sufficient to induce hDF contractility, pro-invasive capacity and MLC2 phosphorylation, and also rescued the inhibitory effect of P6 treatment under TGFβ1 stimulation. Taken together, these data suggest that hDF activation by TGFβ1 or LIF requires acto-myosin contractility, which is regulated by JAK signalling. In conclusion, in fibroblasts, the pro-inflammatory cytokine LIF mediates TGFβ1-dependent acto-myosin contractility and pro-invasive ECM remodelling.

LIF Mediates Fibroblast Activation to Promote Invasive Tumour Microenvironment Independent of Alpha-Smooth Muscle Actin Expression:

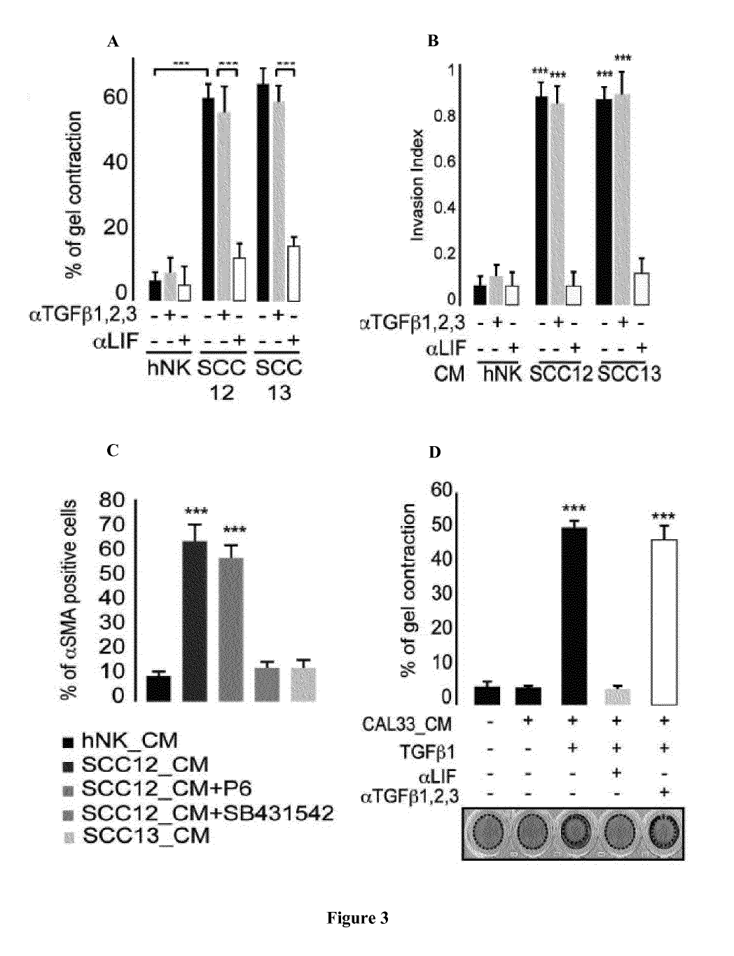

Within the tumour microenvironment, secretion of growth factors and cytokines³¹⁻³⁴ by cancer cells is thought to activate the adjacent fibroblasts^{2,12,13,22}. In our experiments, media conditioned (CM) by SCC12 human carcinoma cell promoted both paracrine STAT3 and SMAD2 activation in fibroblasts, while SCC13 cell CM activated STAT3 but not SMAD2 phosphorylation (Table 1). SCC12 and SCC13 cells were further used to investigate the role of TGFβ/SMAD2 and JAK/STAT3 signalling in tumour cell-dependent pro-invasive fibroblast activation and expression of αSMA protein, the latter being a CAF hallmark activated in hDF by the TGFβ signalling²¹. Both SCC12 and SCC13 CM promoted fibroblasts-dependent collagen gel contraction and collective invasion of SCC12 cells in vitro (FIGS. 3A, 3B and S3Aa-c) compared with hNK CM as negative control (FIG. 3B). Blockade of paracrine SMAD2 phosphorylation by an anti-TGFβ antibody to SCC12 CM had no effect on pro-invasive fibroblast activation (FIG. 3B), whereas addition of either a specific LIF-blocking antibody (FIG. 3B) or the ROCK inhibitor Y27632 completely abrogated the action of SCC12 and SCC13 CM. Importantly, αSMA expression, which depends on TGFβ signalling independently of LIF, was induced by the SCC12 CM (FIG. 3C) but not by the SCC13 CM that activates fibroblasts STAT3 only through LIF secretion (FIG. 3C). Both SCC12 and SCC13 CM increased MLC2 protein levels and activity, which specifically depended on LIF secretion. Thus, in hDF αSMA upregulation appears to be uncoupled from acquisition of pro-invasive capacity, which, on the contrary, is conferred by the “tumoral” LIF that relies on the crosstalk between JAK/STAT and Rho/ROCK/MLC2 signalling pathways. This hypothesis was verified using the CAL33 cell line whose CM neither contains LIF nor activates hDF pro-invasiveness (FIG. 3D and S3G and Table1). Indeed, hDF maintained in CM from CAL33 cells engineered to secrete transgenic hLIF (CAL33-LIF), showed paracrine activation of matrix remodelling and STAT3 phosphorylation (Figure S3F). Also, CAL33-LIF CM induced pro-invasive fibroblast conversion that was blocked by addition of either a LIF-specific blocking antibody or the Y27632 ROCK inhibitor. Because, TGFβ was found to stimulate LIF production in hDF, the capacity of TGFβ-dependent signalling to induce LIF secretion by tumour cells was investigated. Stimulation of CAL33 cells by TGFβ1 resulted in LIF secretion, which promoted both STAT3 activation and contractility in hDF (FIG. 3D). Moreover, addition of a LIF-specific blocking antibody to CM of TGFβ1-stimulated CAL33 resulted in complete inhibition of both STAT3 phosphorylation and matrix remodelling (FIG. 3D). We thus concluded that in human tumours, including skin, head-and-neck, lung, colon and breast carcinomas as well as melanomas (Table 1), LIF signalling mediates onset of a pro-invasive microenvironment by pro-invasive fibroblasts activation and acto-myosin contractility regulation independent of αSMA expression.

TABLE 1
LIF production by carcinoma cells from different origins
mediates pro-invasive fibroblasts activation.

| | STAT3- | SMAD2- | Fibroblasts contractile & pro-invasive | | | | | | | |
|---|---|---|---|---|---|---|---|---|---|---|
| | Y705 | S465/467 | phenotype** | | | | | | | |
| Cancer cell | In | in | | SB | P | | αIL6 | αLIF | αTGFβ | LIF ELISA |
| lines | fibroblasts* | fibroblast* | Veh. | 431 | 6 | Ruxo. | (10 μg · ml⁻¹) | (10 μg · ml⁻¹)‡ | (10 μg · ml⁻¹) | (pg · ml⁻¹)*** |

Human OSCC

| | | | | | | | | | | |
|---|---|---|---|---|---|---|---|---|---|---|
| hPK | − | − | − | − | − | − | − | − | − | UD |
| SCC12 | ++ | ++ | + | + | − | − | + | − | + | 129.05 ± 12.4 |
| SCC13 | ++ | − | + | + | − | − | + | − | + | 276.64 ± 18.6 |
| SCC25 | + | ++ | + | + | − | − | + | − | + | 48.11 ± 3.05 |
| CAL27 | ++ | ++ | + | + | − | − | + | −(50) | + | 400.06 ± 22.12 |
| CAL33 | − | − | − | − | − | − | − | − | − | UD |
| CAL60 | ++ | + | + | + | − | − | + | − | + | 77.33 ± 4.24 |
| CAL166 | ++ | + | + | + | − | − | + | − | + | 84.14 ± 6.32 |
| Detroit562 | + | ++ | + | + | − | − | + | − | + | 102.97 ± 8.43 |

h. Colon Carcinoma

| | | | | | | | | | | |
|---|---|---|---|---|---|---|---|---|---|---|
| LS174 | ++ | + | + | + | − | − | + | − | + | 185.29 ± 9.87 |

h. Lung Carcinoma

| | | | | | | | | | | |
|---|---|---|---|---|---|---|---|---|---|---|
| A549 | ++ | + | + | + | − | − | + | − | + | 95.11 ± 6.49 |

Breast Carcinoma

| | | | | | | | | | | |
|---|---|---|---|---|---|---|---|---|---|---|
| MDA-MB-231 | +++ | ++ | + | + | − | − | +/− | +/− | + | 216.79 ± 15.32 |
| MDA-MB-468 | ++ | ++ | + | + | − | − | + | + | + | 64.34 ± 4.54 |

h. Melanoma

| | | | | | | | | | | |
|---|---|---|---|---|---|---|---|---|---|---|
| A375P | ++ | + | + | + | − | − | + | − | + | 276.69 ± 18.12 |
| A375M2 | +++ | ++ | + | + | − | − | + | −(50) | + | 545.38 ± 32.87 |
| Mel501 | − | − | − | − | − | − | − | − | − | UD |
| Sbcl2 | − | − | − | − | − | − | N.D. | − | N.D. | 6.78 ± 2.32 |
| WM35 | ++ | − | + | + | − | − | N.D. | − | N.D. | 403 ± 30.32 |
| WM278 | + | + | + | + | − | − | N.D. | − | N.D. | 98.07 ± 7.3 |
| WM793 | + | + | + | + | − | − | N.D. | − | N.D. | 251.13 ± 13 |
| Mel1205 | ++ | N.D. | + | + | − | − | N.D. | − | N.D. | 198.46 ± 65 |

h. Engineered Cell line

| | | | | | | | | | | |
|---|---|---|---|---|---|---|---|---|---|---|
| CAL33_mock | − | − | − | − | − | − | − | − | − | UD |
| CAL33_LIF | +++ | − | + | + | − | − | + | +(50) | + | 1176.03 ± 86.5 |

Murine Breast Carcinoma

| | | | | | | | | | |
|---|---|---|---|---|---|---|---|---|---|
| 67NR | − | − | − | N.D. | − | N.D. | − | N.D. | 14.5 ± 20.51 |
| 4T07 | ++ | +/− | + | N.D. | − | N.D. | − | N.D. | 603.09 ± 49.54 |
| 410.4 | ++ | ++ | + | N.D. | − | N.D. | − | N.D. | 927.35 ± 17.00 |
| 4T1 | ++ | + | + | N.D. | − | N.D. | − | N.D. | 1523.25 ± 162.8 |

*Tumour cell conditioned media (CM) induces STAT3 and SMDA2 transcription factors activation in fibroblasts was determined by Western blot (shown in Supplementary FIG. 6). Phosphorylation detectable (+) or undetectable (−).
**Both contractility and pro-invasive activities induced in fibroblasts by tumour cell CM were determined using three-dimensional collagen lattices and organotypic invasion assays. +: induction, −: no induction.
***LIF was detected by ELISA method.
‡LIF and TGFβ blocking antibodies were used at 10 μg · ml−1 or as stated.
N.D. not determined

LIF and JAK Kinase Signalling Drive Invasive Tumour Microenvironment in Breast Carcinomas:

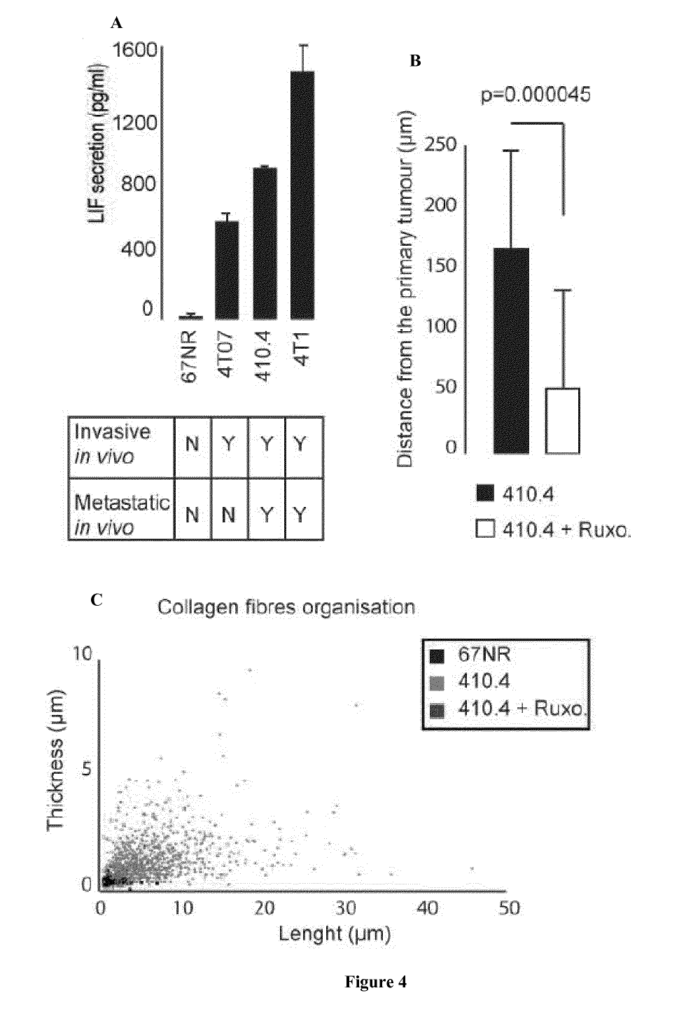

The role of LIF production by tumour cells during invasive tumour ECM remodelling was then investigated in vivo. LIF secretion was first monitored in a panel of mouse breast carcinoma cell lines spanning from a poorly tumorigenic to a highly invasive phenotype^{35,36.} High in-vitro LIF secretion levels were found with 4T07, 410.4 and 4T1 invasive cancer cells, while LIF secretion was low in 67NR non-invasive tumour cells (FIG. 4A). In vitro, LIF production by mouse tumour cells correlated with potential to induce contractility in mouse fibroblasts. LIF low-producer (67NR) and LIF high-producer (410.4) mouse breast carcinoma cells were then injected into mammary fat pads of syngeneic BALB/c female mice. 30 days after implantation, mice were sacrificed and primary tumours were analysed by immunohistology. Strong LIF-specific staining was exclusively observed in the primary tumour mass generated by 401.4 cells that correlated with sustained STAT3 activation in 410.4 but not in 67NR tumours both in vivo and in vitro, which identify in LIF secreted by the tumour cells the major cytokine driving STAT3 activation in fibroblasts in vivo. As expected, in vitro stimulation of mouse dermal fibroblasts by CM of 410.4 cells resulted in SMAD2 activation and αSMA expression, while 67NR CM failed to activate SMAD2 and STAT3 and to induce αSMA expression. In vivo, αSMA expression was detected within the tumour mass of 401.4 tumour microenvironment but not within 67NR tumour, suggesting a strong correlation between the in vitro and in vivo situations. In tumours, collagen fibres are indicative of a dense ECM and correlates with the invasive potential of tumour cells³⁷⁻³⁹. Assessment of collagen production and collagen fibres assembly in xenograft tumours by Sirius Red staining disclosed enhanced decoration in 410.4 tumours compared with 67NR tumours, with formation of polarized collagen fibres (FIG. 4C).

To disclose the fundamental role of JAK kinase activity in fibroblasts-mediated pro-invasive ECM remodelling, we further assessed the effect of the JAK1/2 inhibitor Ruxolitinib on tumour microenvironment development in vivo by 21-day oral gavage of mice implanted with 401.4 cells. The drug inhibited STAT3 phosphorylation both within the tumour microenvironment in vivo and in vitro, without influencing LIF production or αSMA staining. JAK signalling inhibition reduced Sirius Red staining and formation of collagen bundles (FIG. 4C), which underscores the role of JAK in collagen fibres assembly within the tumour microenvironment in vivo. Consistent with the fact that matrix stiffness promotes tumour cell invasion in vitro and in vivo^{37,38,40,41}, we disclosed that in mice treated with Ruxolitinib the tumour cells displayed a significant reduced rates of invasion (FIG. 4B).

We next unveiled the collaborative role of both TGFβ and JAK signalling during ECM remodelling in vitro. In hDF, and opposite to LIF stimulation, fibronectin expression pattern was upregulated by TGFβ1 stimulation and slightly reduced after addition of P6 inhibitor. Moreover, TGFβ1-stimulated hDF exhibited an enhanced fibronectin secretion and deposition into the matrix compared to LIF-stimulated and control hDF. Further, JAK inhibition was ineffective on fibronectin secretion but dramatically reduced its assembly into the matrix, which discloses a collaborative role of TGFβ1 and JAK signalling in fibroblast-dependent ECM remodelling and matrix protein assembly. We also demonstrated that TGFβ1-stimulated mouse dermal fibroblasts secrete LIF in the culture media, which leads to collagen gel contraction in vitro and in autocrine activation of STAT3 phosphorylation. Blockade of JAK kinase activity by either addition of Ruxolitinib or LIF sequestration using a specific blocking antibody resulted in both complete abrogation of STAT3 phosphorylation and matrix remodelling. Thus, the mice xenograft breast cancer model demonstrates that LIF, independently of αSMA expression, supports tumour stroma remodelling and that Ruxolitinib by inhibiting JAK abrogates the pro-invasive crosstalk between tumour and stroma cells both in vitro and in vivo.

LIF is Overexpression in Human Skin Carcinoma and Correlates with Invasive Tumour Microenvironment:

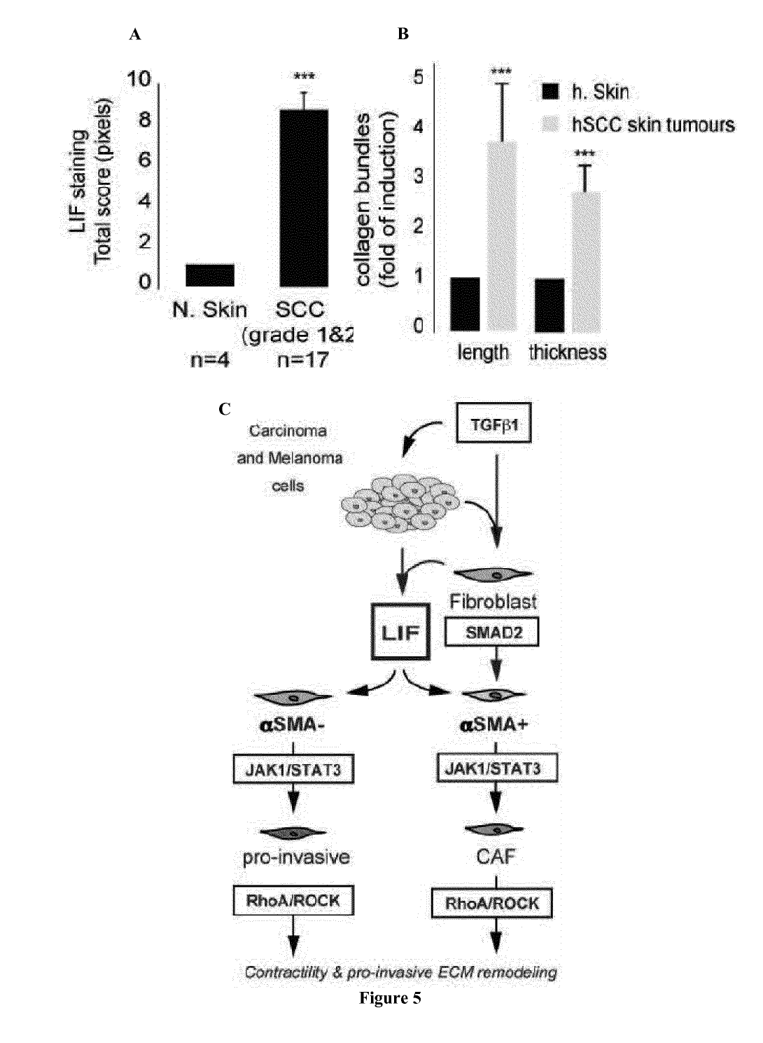

Randomized analysis of 17 skin biopsies from human SCC grade 1 to 2 (sup. Table 2) detected strong diffused LIF staining compared to control skin where LIF expression is confined to basal keratinocytes (FIG. 5A). LIF up-regulation in carcinomas correlated with marked assembly of collagen bundles (FIG. 5B). Collagen bundle formation by hDF was verified in vitro using 3D-organotypic cultures. hDF stimulated by either TGFβ1 or LIF showed an enhanced capacity to assembly polarized collagen fibres which was inhibited by Ruxolitinib, thus providing further evidence for the role of fibroblast JAK kinase in tumour ECM remodelling. To confirm the results obtained in mice, the inhibitory effect of Ruxolitinib on collective carcinoma cell invasion was assessed in vitro. The drug prevented track formation and SCC12 cell invasion promoted by hCAF through inhibition of the CAF endogenous STAT3 activity. Moreover, Ruxolitinib completely inhibited both TGFβ1 and LIF-dependent activation of hDF contractility in collagen rich lattices) and abrogated the pro-invasive hDF activation induced upon TGFβ1 and LIF stimulation by repressing STAT3 phosphorylation in TGFβ1- or LIF-stimulated fibroblasts with no effect on SMAD2. Therefore, use of the Ruxolitinib inhibitor of JAK1/2 kinases may pave the way to therapeutic approaches aiming at thwarting not only cancer invasion, but also stroma activation during the early steps of aggressive carcinoma development.

SUPLEMENTAL TABLE 2
List and designation of human skin SCC biological
specimen and their respective quantification for LIF
himmunohistological staining.

| Patient | Ref. | Grade | I score | P score | Total score |
|---|---|---|---|---|---|
| #1 | 9-7703 | I | 2 | 2 | 4 |
| #2 | 8-29879 | I | 3 | 1 | 3 |
| #3 | 9-14512A | I | 3 | 4 | 12 |
| #4 | 8-42310 | I | 2 | 3 | 6 |
| #5 | 9-5297 | I | 2 | 1 | 2 |
| #6 | 9-5696 | I | 2 | 2 | 4 |
| #7 | 9-6203A | II | 3 | 4 | 12 |
| #8 | 8-31231 | I | 3 | 4 | 12 |
| #9 | 8-35975 | I | 1 | 1 | 1 |
| #10 | 8-37896 | I | 3 | 2 | 6 |
| #11 | 9-16767 | I | 2 | 1 | 2 |
| #12 | 9-5302 | I | 3 | 1 | 3 |
| #13 | 9-5301 | I | 1 | 4 | 4 |
| #14 | 9-1499A | I | 3 | 4 | 12 |
| #15 | 8-37899 | I | 3 | 4 | 12 |
| #16 | 9-1052 | II | 3 | 2 | 6 |
| #17 | 8-38005 | I | 2 | 4 | 8 |

Summary of the results obtained with immunohistochemistry staining for LIF in human skin SCC from grade I and II specimen scored by himmunohistopathologists. I score=Intensity of staining (0-negative; 1-weak; 2-moderate; 3-strong), P score=Percentage of positive staining (0<10% of positive signal; 10%<1<25%; 26%<2<50%; 51%<3<75%; 76%<4<100%), Total score=P×I for each section. Quantification was done in a double-blinded method.

### DISCUSSION

We identify LIF, a member of the IL6 pro-inflammatory cytokine family, as the main driver of pro-invasive TGFβ-dependent evolution of the tumour microenvironment. LIF mediates autocrine TGFβ1-dependent pro-invasive activation in fibroblasts, while, in a paracrine manner, tumour-secreted LIF promotes and sustains pro-invasive conversion of fibroblast independent of αSMA expression (FIG. 5C). LIF is overexpressed in a variety of solid tumors including skin cancers^{32,33} and tumor cells LIF production correlates with their invasive potential^{35,42}. However, down-regulation of LIFr recently reported in cancer cells⁴³ prompted the idea that tumor cell-derived LIF may exert paracrine effect in tumorigenesis. We propose that paracrine activities of LIF are related to stromal fibroblast pro-invasive activation. CAF are key players of cancer-associated inflammatory processes^{6,17}. Secretion of IL11 upon TGFβ stimulation of CAF was shown to induce GP130-IL6ST/STAT3 signalling in colorectal cancer, which confers a survival advantage to metastatic cells and leads to increased efficiency of organ colonization⁴⁴. Our results, showing that TGFβ1-mediated LIF secretion both in tumor cells and fibroblasts, emphasize the action of TGFβ1 as a driver of tumor-associated inflammation, which is a key step for stromal fibroblast pro-invasive activation. LIF transcription regulation by TGFβ1 was recently implicated in glioblastoma tumor-initiating cell renewal⁴⁵, which suggests a general regulation of LIF by TGFβ1 in cancer.

αSMA expression, which is hallmark of CAF¹³ is regulated by TGFβ-dependent signalling²¹, regulates fibroblasts contractility⁴⁶ and correlates with poor clinical outcome in human tumours⁴⁷. Here, we provide evidence that LIF-stimulated dermal fibroblasts, independent of αSMA expression, promote collagen gel contractility in vitro and that blockade of JAK activity in vivo results in inhibition of tumour-associated collagen network. LIF-activated dermal fibroblast acquires contractile phenotype via a crosstalk between the JAK1/STAT3 and RhoA/ROCK/MLC2 signalling pathways, suggesting that αSMA and Rho-dependent contractility may be regulated by two distinct mechanisms both resulting in cell contractility and ECM remodelling. Within the tumour microenvironment, hCAF are highly heterogeneous and αSMA is not expressed by all hCAF cells⁴⁸. Moreover, PDGFRα has been proposed to be a robust marker for CAF in a mice model of skin cancer¹⁶. Similarly, we noted an increased expression of PDGFRα after LIF stimulation in hPDF (data not shown). In light of our results, we propose that LIF generates subpopulations of CAF cells prone to malignancy independently of αSMA expression. This implies that αSMA expression is not sufficient to disclose presence of all pro-invasive fibroblasts within the tumour stroma, which thus may lead to biased favourable prognosis for patients.

Acto-myosin contractility is crucial for CAF-dependent pro-invasive matrix remodelling¹⁸. We now provide direct evidence that LIF supports TGFβ1-dependent acto-myosin contractility in hPDF. Accordingly, formation of collagen fibres in tumours is indicative of ECM densification and correlates with acquisition of invasive potential by tumour cells³⁷⁻³⁹. Similar to human SCC tumours overexpressing LIF, in vivo orthotopic mouse breast cancer model, and hDF in vitro cell cultures, disclose that LIF and JAK drive TGFβ1-induced dense collagen fibres remodelling in vivo and in vitro through RhoA and MLC2 overexpression in hDF, which results in increased MLC2 phosphorylation and acto-myosin contractility. Acto-myosin contractility induces matrix stiffening³⁹ and, conversely, stiff matrix activates YAP/TAZ and RhoA/ROCK-dependent signalling pathways in CAF³ and favors tumour cell invasion^{3,37,38,40,41}. We now show that forced expression of a constitutively activated ROCK protein is sufficient to induce pro-invasive fibroblasts activity in vitro. This loop may constitute a mechanism by which hPDF may also be activated during tumourigenesis³. In this context, TGFβ signalling drives fibronectin extracellular matrix protein expression, while JAK1 kinase activity, through acto-myosin contractility, is responsible for protein assembly into the matrix, which suggests a collaborative role for both TGFβ and JAK signalling pathways in tumour fibrosis. This finding is in agreement with the fact that, JAK2 drives the profibrotic effect of TGFβ signalling in systemic sclerosis and cutaneous fibrosis^{49,50}, a process also implying STAT3 in lung fibrosis⁵¹. Because fibrosis is known to be mediated by excessive ECM remodelling and collagen fibre assembly⁵², it is conceivable that the JAK1/STAT3 signalling route supports the hCAF pro-fibrotic activity in cancer, which results in a matrix prone to collective cancer cell invasion. In light of our data, we propose that, via still undefined mechanisms, constitutive activation of STAT3, may occur in hCAF which influences invasiveness of human cancers. Accordingly, blockade of JAK activity by the JAK1/2 inhibitor Ruxolitinib counteracts the TGFβ and LIF-mediated fibroblast-dependent collective carcinoma cell invasion in vitro and in vivo, Ruxolitinib has been approved by the Food and Drug Administration for treatment of patients with intermediate or high-risk myelofibrosis⁵³, and this drug is also in a phase II clinical trial for patients with breast or pancreatic cancer in which STAT3 is frequently constitutively activated^{54,55}. Therefore, clinical use of the JAK1/2 Ruxolitinib inhibitor may be a promising approach in combination with chemotherapies for patients suffering from aggressive cancers through the targeting of stromal fibroblasts and ECM-associated remodelling events.

In conclusion, our work reveals a novel role of LIF in malignancy. LIF signalling in fibroblasts results in pro-invasive tumour microenvironment promotion and mediates TGFβ-dependent fibrosis in cancer. We speculate that blockade of JAK kinase activity within the tumour mass may constitute a promising therapeutic approach to counteract the CAF-dependent pro-tumorigenic ECM remodelling.

### REFERENCES

Throughout this application, various references describe the state of the art to which this invention pertains. The disclosures of these references are hereby incorporated by reference into the present disclosure.
1 Hanahan, D. & Weinberg, R. A. Hallmarks of cancer: the next generation. Cell 144, 646-674, doi:S0092-8674(11)00127-9 [pii]
   10.1016/j.cell.2011.02.013 (2011).
2 Calvo, F. & Sahai, E. Cell communication networks in cancer invasion. Curr Opin Cell Biol 23, 621-629, doi:S0955-0674(11)00058-5 [pii]
   10.1016/j.ceb.2011.04.010 (2011).
3 Calvo, F. et al. Mechanotransduction and YAP-dependent matrix remodelling is required for the generation and maintenance of cancer-associated fibroblasts. Nat Cell Biol, doi:10.1038/ncb2756
   ncb2756 [pii](2013).
4 Joyce, J. A. & Pollard, J. W. Microenvironmental regulation of metastasis. Nat Rev Cancer 9, 239-252, doi:nrc2618 [pii]
   10.1038/nrc2618 (2009).
5 Boyd, N. F. et al. Mammographic density and the risk and detection of breast cancer. N Engl J Med 356, 227-236, doi:356/3/227 [pii]
   10.1056/NEJMoa062790 (2007).
6 Navab, R. et al. Prognostic gene-expression signature of carcinoma-associated fibroblasts in non-small cell lung cancer. Proc Natl Acad Sci USA 108, 7160-7165, doi:1014506108 [pii]
   10.1073/pnas.1014506108 (2011).
7 Lin, X. D. et al. Overexpression of thrombospondin-1 in stromal myofibroblasts is associated with tumor growth and nodal metastasis in gastric carcinoma. J Surg Oncol 106, 94-100, doi:10.1002/jso.23037 (2012).
8 Shi, M. et al. Expression of fibroblast activation protein in human pancreatic adenocarcinoma and its clinicopathological significance. World J Gastroenterol 18, 840-846, doi:10.3748/wjg.vl8.i8.840 (2012).
9 Takahashi, Y. et al. Fibrous stroma is associated with poorer prognosis in lung squamous cell carcinoma patients. J Thorac Oncol 6, 1460-1467, doi:10.1097/JTO.0b013e318229189d
   01243894-201109000-00004 [pii](2011).
10 Beacham, D. A. & Cukierman, E. Stromagenesis: the changing face of fibroblastic microenvironments during tumor progression. Semin Cancer Biol 15, 329-341, doi:S1044-579X(05)00027-1 [pii]
   10.1016/j.semcancer.2005.05.003 (2005).
11 Bhowmick, N. A., Neilson, E. G. & Moses, H. L. Stromal fibroblasts in cancer initiation and progression. Nature. 432, 332-337. (2004).
12 De Wever, O., Demetter, P., Mareel, M. & Bracke, M. Stromal myofibroblasts are drivers of invasive cancer growth. Int J Cancer 123, 2229-2238, doi:10.1002/ijc.23925 (2008).
13 Kalluri, R. & Zeisberg, M. Fibroblasts in cancer. Nat Rev Cancer. 6, 392-401. (2006).
14 Olumi, A. F. et al. Carcinoma-associated fibroblasts direct tumor progression of initiated human prostatic epithelium. Cancer Res. 59, 5002-5011. (1999).
15 Trimboli, A. J. et al. Pten in stromal fibroblasts suppresses mammary epithelial tumours. Nature. 461, 1084-1091. (2009).
16 Erez, N., Truitt, M., Olson, P., Arron, S. T. & Hanahan, D. Cancer-Associated Fibroblasts Are Activated in Incipient Neoplasia to Orchestrate Tumor-Promoting Inflammation in an NF-kappaB-Dependent Manner. Cancer Cell 17, 135-147. Epub 210 February 2014. (2010).
17 Orimo, A. et al. Stromal fibroblasts present in invasive human breast carcinomas promote tumor growth and angiogenesis through elevated SDF-1/CXCL12 secretion. Cell. 121, 335-348. (2005).
18 Gaggioli, C. et al. Fibroblast-led collective invasion of carcinoma cells with differing roles for RhoGTPases in leading and following cells. Nat Cell Biol. 9, 1392-1400. Epub 27 Nov. 1325. (2007).
19 Coussens, L. M. & Werb, Z. Inflammation and cancer. Nature. 420, 860-867. (2002).
20 Lederle, W. et al. IL-6 promotes malignant growth of skin SCCs by regulating a network of autocrine and paracrine cytokines. Int J Cancer 128, 2803-2814, doi:10.1002/ijc.25621 (2011).
21 Desmouliere, A., Geinoz, A., Gabbiani, F. & Gabbiani, G. Transforming growth factor-beta 1 induces alpha-smooth muscle actin expression in granulation tissue myofibroblasts and in quiescent and growing cultured fibroblasts. J Cell Biol 122, 103-111 (1993).
22 Phan, S. H. Biology of fibroblasts and myofibroblasts. Proc Am Thorac Soc. 5, 334-337. (2008).
23 Bhowmick, N. A. et al. TGF-beta signaling in fibroblasts modulates the oncogenic potential of adjacent epithelia. Science. 303, 848-851. (2004).
24 Franco, O. E. et al. Altered TGF-beta signaling in a subpopulation of human stromal cells promotes prostatic carcinogenesis. Cancer Res 71, 1272-1281, doi:0008-5472.CAN-10-3142 [pii]
   10.1158/0008-5472.CAN-10-3142 (2011).
25 Achyut, B. R. et al. Inflammation-Mediated Genetic and Epigenetic Alterations Drive Cancer Development in the Neighboring Epithelium upon Stromal Abrogation of TGF-beta Signaling. PLoS Genet 9, e1003251, doi: 10.1371/journal.pgen.1003251
   PGENETICS-D-12-02092 [pii](2013).
26 Mordasky Markell, L., Perez-Lorenzo, R., Masiuk, K. E., Kennett, M. J. & Glick, A. B. Use of a TGFbeta type I receptor inhibitor in mouse skin carcinogenesis reveals a dual role for TGFbeta signaling in tumor promotion and progression. Carcinogenesis 31, 2127-2135, doi:bgq191 [pii]
   10.1093/carcin/bgq191 (2010).
27 Connolly, E. C., Freimuth, J. & Akhurst, R. J. Complexities of TGF-beta targeted cancer therapy. Int J Biol Sci 8, 964-978, doi:10.7150/ijbs.4564
   ijbsv08p0964 [pii](2012).
28 Sanz-Moreno, V. et al. ROCK and JAK1 signaling cooperate to control actomyosin contractility in tumor cells and stroma. Cancer Cell 20, 229-245, doi:S1535-6108(11)00230-3 [pii]
   10.1016/j.ccr.2011.06.018 (2011).
29 Kishimoto, T., Akira, S., Narazaki, M. & Taga, T. Interleukin-6 family of cytokines and gp130. Blood. 86, 1243-1254. (1995).
30 Croft, D. R. & Olson, M. F. Conditional regulation of a ROCK-estrogen receptor fusion protein. Methods Enzymol 406, 541-553, doi:S0076-6879(06)06042-3 [pii]
   10.1016/S0076-6879(06)06042-3 (2006).
31 Szepietowski, J. C., Reich, A. & McKenzie, R. C. The multifunctional role of leukaemia inhibitory factor in cutaneous biology. Acta Dermatovenerol Alp Panonica Adriat 13, 125-129, doi:00000003 [pii](2004).
32 Garcia-Tunon, I. et al. OSM, LIF, its receptors, and its relationship with the malignance in human breast carcinoma (in situ and in infiltrative). Cancer Invest 26, 222-229, doi:790002031 [pii]
   10.1080/07357900701638491 (2008).
33 Wysoczynski, M. et al. Leukemia inhibitory factor: a newly identified metastatic factor in rhabdomyosarcomas. Cancer Res 67, 2131-2140, doi:67/5/2131 [pii]
   10.1158/0008-5472.CAN-06-1021 (2007).
34 Sriuranpong, V. et al. Epidermal growth factor receptor-independent constitutive activation of STAT3 in head and neck squamous cell carcinoma is mediated by the autocrine/paracrine stimulation of the interleukin 6/gp130 cytokine system. Cancer Res 63, 2948-2956 (2003).
35 Aslakson, C. J. & Miller, F. R. Selective events in the metastatic process defined by analysis of the sequential dissemination of subpopulations of a mouse mammary tumor. Cancer Res 52, 1399-1405 (1992).
36 Yang, J. et al. Twist, a master regulator of morphogenesis, plays an essential role in tumor metastasis. Cell 117, 927-939, doi:10.1016/j.cell.2004.06.006 S0092867404005768 [pii](2004).
37 Paszek, M. J. et al. Tensional homeostasis and the malignant phenotype. Cancer Cell 8, 241-254, doi:S1535-6108(05)00268-0 [pii]
   10.1016/j.ccr.2005.08.010 (2005).
38 Levental, K. R. et al. Matrix crosslinking forces tumor progression by enhancing integrin signaling. Cell. 139, 891-906. (2009).
39 Samuel, M. S. et al. Actomyosin-mediated cellular tension drives increased tissue stiffness and beta-catenin activation to induce epidermal hyperplasia and tumor growth. Cancer Cell 19, 776-791, doi:10.1016/j.ccr.2011.05.008
   S1535-6108(11)00166-8 [pii](2011).
40 Goetz, [40] J. G. et al. Biomechanical remodeling of the microenvironment by stromal caveolin-1 favors tumor invasion and metastasis. Cell 146, 148-163, doi:10.1016/j.cell.2011.05.040
   S0092-8674(11)00645-3 [pii](2011).
41 Provenzano, P. P. et al. Collagen density promotes mammary tumor initiation and progression. BMC Med. 6, 11. (2008).
42 Clark, E. A., Golub, T. R., Lander, E. S. & Hynes, R. O. Genomic analysis of metastasis reveals an essential role for RhoC. Nature 406, 532-535, doi:10.1038/35020106 (2000).
43 Chen, D. et al. LIFR is a breast cancer metastasis suppressor upstream of the Hippo-YAP pathway and a prognostic marker. Nat Med 18, 1511-1517, doi:10.1038/nm.2940 nm.2940 [pii](2012).
44 Calon, A. et al. Dependency of Colorectal Cancer on a TGF-beta-Driven Program in Stromal Cells for Metastasis Initiation. Cancer Cell 22, 571-584, doi: 10.1016/j.ccr.2012.08.013
   S1535-6108(12)00355-8 [pii](2012).
45 Penuelas, S. et al. TGF-beta increases glioma-initiating cell self-renewal through the induction of LIF in human glioblastoma. Cancer Cell 15, 315-327, doi:S1535-6108(09)00042-7 [pii]
   10.1016/j.ccr.2009.02.011 (2009).
46 Hinz, B., Celetta, G., Tomasek, J. J., Gabbiani, G. & Chaponnier, C. Alpha-smooth muscle actin expression upregulates fibroblast contractile activity. Mol Biol Cell 12, 2730-2741 (2001).
47 Yamashita, M. et al. Role of stromal myofibroblasts in invasive breast cancer: stromal expression of alpha-smooth muscle actin correlates with worse clinical outcome. Breast Cancer 19, 170-176, doi:10.1007/s12282-010-0234-5 (2012).
48 Sugimoto, H., Mundel, T. M., Kieran, M. W. & Kalluri, R. Identification of fibroblast heterogeneity in the tumor microenvironment. Cancer Biol Ther 5, 1640-1646, doi:3354 [pii](2006).
49 Dees, C. et al. JAK-2 as a novel mediator of the profibrotic effects of transforming growth factor beta in systemic sclerosis. Arthritis Rheum 64, 3006-3015, doi:10.1002/art.34500 (2012).
50 Canady, J., Arndt, S., Karrer, S. & Bosserhoff, A. K. Increased KGF Expression Promotes Fibroblast Activation in a Double Paracrine Manner Resulting in Cutaneous Fibrosis. J Invest Dermatol, doi: 10.1038/jid.2012.389
   jid2012389 [pii](2012).
51 O'Donoghue, R. J. et al. Genetic partitioning of interleukin-6 signalling in mice dissociates Stat3 from Smad3-mediated lung fibrosis. EMBO Mol Med 4, 939-951, doi:10.1002/emmm.201100604 (2012).
52 Wynn, T. A. & Ramalingam, T. R. Mechanisms of fibrosis: therapeutic translation for fibrotic disease. Nat Med 18, 1028-1040, doi:10.1038/nm.2807 nm.2807 [pii](2012).
53 Mascarenhas, J. & Hoffman, R. Ruxolitinib: the first FDA approved therapy for the treatment of myelofibrosis. Clin Cancer Res 18, 3008-3014, doi:1078-0432.CCR-11-3145 [pii]10.1158/1078-0432.CCR-11-3145 (2012).
54 Berishaj, M. et al. Stat3 is tyrosine-phosphorylated through the interleukin-6/glycoprotein 130/Janus kinase pathway in breast cancer. Breast Cancer Res 9, R³², doi:bcr1680 [pii]
   10.1186/bcr1680 (2007).
55 Scholz, A. et al. Activated signal transducer and activator of transcription 3 (STAT3) supports the malignant phenotype of human pancreatic cancer. Gastroenterology 125, 891-905, doi:S0016508503010643 [pii](2003).
56 Rheinwald, J. G. & Green, H. Serial cultivation of strains of human epidermal keratinocytes: the formation of keratinizing colonies from single cells. Cell 6, 331-343 (1975).
57 Hooper, S., Gaggioli, C. & Sahai, E. A chemical biology screen reveals a role for Rab21-mediated control of actomyosin contractility in fibroblast-driven cancer invasion. Br J Cancer 102, 392-402 (2009).

1. A method of treating or preventing metastasis in a patient affected with a Leukemia Inhibitory Factor (LIF)-expressing solid cancer, comprising administering to the patient a therapeutically effective amount of an inhibitor of the Janus Kinase 1 (JAK1)/Signal Transducer and Activator of Transcription 3 (STAT3) signalling pathway.

2. The method of claim 1, wherein said inhibitor is 3-Cyclopentyl-3-[4-(7H-pyrrolo[2,3-d]pyrimidin-4-yl)-1H-pyrazol-1-yl]propanenitrile (ruxolitinib) or a pharmaceutically acceptable salt thereof.

3. The method of claim 1, wherein said inhibitor is a JAK1 selective inhibitor.

4. A method of inhibiting the pro-invasive stromal fibroblast activity of carcinoma-associated fibroblasts (CAFs) in a patient affected with a LIF-expressing solid cancer, comprising administering to the patient a therapeutically effective amount of an inhibitor of the JAK1/STAT3-signalling pathway.

5. A method of inhibiting the pro-invasive stromal fibroblast activation of carcinoma-associated fibroblasts (CAFs) in a patient affected with a LIF-expressing solid cancer, comprising administering to the patient a therapeutically effective amount of a LIF antagonist.

6. The method of claim 5 wherein said antagonist is an anti-LIF antibody, an anti-Glycoprotein 130 (GP130) antibody or an anti-LIF receptor (LIFR) antibody.

7. The method of claim 1, wherein the LIF-expressing solid cancer is an epithelial cancer or melanoma.

8. The method of claim 7, wherein the epithelial cancer is selected from the group consisting of skin, head-and-neck, lung, colon and breast carcinomas.

9. A method for determining whether a patient is likely to benefit from a therapy with an inhibitor of the JAK1/STAT3 signalling pathway and/or a LIF antagonist, comprising a step of (i) determining an expression level of LIF in a biological sample obtained from said patient, and (ii) comparing said expression level with a predetermined reference value, wherein an increase in expression level of the LIF gene is indicative of metastasis or of a risk of metastasis, and that the patient is thus likely to benefit form the therapy.

10. The method according to claim 9, wherein the biological sample is a biopsy sample.

11. The method according to claim 9, comprising a further step of determining a level of phospho-Tyr705-STAT3 and/or a fibrotic microenvironment and/or a collective cancer cell pattern of invasion.

12. A method of treating recessive dystrophic epidermolysis bullosa (RDEB) in a patient in need thereof, comprising administering to the subject a therapeutically effective amount of an inhibitor of the JAK1/STAT3 signalling pathway and/or a LIF antagonist.

13. The method of claim 12, wherein administration of the JAK1/STAT3 signalling pathway and/or a LIF antagonist prevents metastasis in the patient.

14. A pharmaceutical composition or a kit-of-parts composition comprising one or more of an inhibitor of the JAK1/STAT3 signalling pathway, a LIF antagonist and a DNA methyltransferase (DNMT) inhibitor.

15. The pharmaceutical composition or a kit-of-parts composition of claim 14, wherein said pharmaceutical composition or a kit-of-parts composition comprises i) an inhibitor of the JAK1/STAT3 signalling pathway and a LIF antagonist, or ii) an inhibitor of the JAK1/STAT3 signalling pathway and a DNMT inhibitor, and is for use in preventing metastasis or for improving survival time in a patient in need thereof.

16-18. (canceled)

19. A The pharmaceutical composition or a kit-of-parts composition of claim 14, wherein the inhibitor of the JAK1/STAT3 signalling pathway is Ruxolitinib and the DNMT inhibitor is Azacytidine.


SEQUENCE LISTING (6 sequences)

SEQ ID NO: 1 - RNA, 19 nt, Artificial
  Synthetic siRNA against JAK1
gacaugauauugagaacga

SEQ ID NO: 2 - RNA, 19 nt, Artificial
  Synthetic siRNA against JAK1
uuacaaggaugacgaagga

SEQ ID NO: 3 - RNA, 19 nt, Artificial
  Synthetic siRNA against STAT3
gagauugaccagcaguaua

SEQ ID NO: 4 - RNA, 19 nt, Artificial
  Synthetic siRNA against STAT3
ccaacaaucccaagaaugu

SEQ ID NO: 5 - RNA, 19 nt, Artificial
  Synthetic siRNA against GP130
cuaauuacauugucuggaa

SEQ ID NO: 6 - RNA, 19 nt, Artificial
  Synthetic siRNA against GP130
cuaaggagcaauauacuau